(19) United States
(12) Patent Application Publication
(10) Pub. No.: US 2025/0009503 A1
(43) Pub. Date: Jan. 9, 2025

(54) PROSTHETIC VALVES WITH VALVULAR STRUCTURES RETAINED BETWEEN TWO FRAMES

(71) Applicant: EDWARDS LIFESCIENCES CORPORATION, Irvine, CA (US)

(72) Inventors: Noa Axelrod Manela, Kadima (IL); Michael Bukin, Pardes Hanna (IL); Noam Nir, Pardes-Hanna (IL); Eran Goldberg, Nesher (IL)

(21) Appl. No.: 18/891,007

(22) Filed: Sep. 20, 2024

Related U.S. Application Data
(63) Continuation of application No. PCT/US2023/015985, filed on Mar. 22, 2023.
(60) Provisional application No. 63/323,021, filed on Mar. 23, 2022.

(51) Int. Cl. A61F 2/24; A61F 2/00; A61F 2/852

(52) U.S. Cl. CPC A61F 2/2418; A61F 2002/0081; A61F 2/852; A61F 2210/0076; A61F 2220/0075

(57) ABSTRACT

The present disclosure relates to prosthetic valves that include valvular structures retained between two frames coupled to each other. In an example, the prosthetic valve includes a main frame, an inner frame which is disposed radially inwardly from, and is coupled to, the main frame, and a valvular structure disposed between the main frame and the inner frame. The main frame is movable between a radially compressed state and a radially expanded state. The inner frame comprises a plurality of struts and a plurality of spikes extending from at least some of the plurality of struts. The plurality of spikes are engaged with the valvular structure.

### CROSS-REFERENCE TO RELATED APPLICATIONS

The present application is a continuation of International Application No. PCT/US2023/015985, filed Mar. 22, 2023, which claims benefit of U.S. Provisional Application No. 63/323,021, filed Mar. 23, 2022, the contents of each of which are herein incorporated by reference in their entirety.

### FIELD

The present invention relates to prosthetic valves, and in particular, to prosthetic heart valves that include valvular structures retained between two frames coupled to each other.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Since surgeries are prone to an abundance of clinical complications, alternative less invasive techniques of delivering a prosthetic heart valve over a catheter and implanting it over the native malfunctioning valve have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

In a typical prosthetic valve, the leaflets of the valve are sutured to an inner skirt that extends along the inner surface of the frame, or sutured directly to struts of the frame. The suturing process tends to be labor-intensive, and the quality of the result may depend on the skill-level of the assembler. Moreover, the tension applied to the suture may not be well controlled, which can effect local geometries of the leaflet. All of these factors may adversely impact the functionality and structural integrity of the leaflets in the long term. Accordingly, improvements to devices and methods for securing leaflets or a leaflet structure to a frame of the valve are desirable.

The present disclosure is directed toward prosthetic heart valves that include an adjustable commissure supports to which leaflet assemblies are attached, wherein the adjustable commissure supports include adjustable arms that can be either rotated or twisted, thereby enabling the tension of the leaflets attached thereto to be adjusted, so as to match the desired expansion diameter of the frame, and allow proper coaptation between the leaflets when the frame is expanded to the desired diameter.

According to some aspects of the disclosure, there is provided a prosthetic valve comprising a main frame, an inner frame which is disposed radially inwardly from, and is coupled to, the main frame, and a valvular structure disposed between the main frame and the inner frame. The main frame comprises a plurality of struts, and is movable between a radially compressed state and a radially expanded state. The inner frame comprises a plurality of struts and a plurality of spikes extending from at least some of the plurality of struts. The plurality of spikes are engaged with the valvular structure.

In some examples, the valvular structure comprises a movable portion and an engagement portion, wherein the movable portion is configured to regulate flow through the prosthetic valve.

In some examples, the plurality of spikes are engaged with the engagement portion of the valvular structure.

According to some aspects of the disclosure, there is provided a prosthetic valve comprising a main frame, an inner frame which is disposed radially inwardly from, and is coupled to, the main frame, and a valvular structure disposed between the main frame and the inner frame. The main frame comprises a plurality of struts defining a plurality of rungs of struts, and is movable between a radially compressed state and a radially expanded state. The inner frame comprises a plurality of struts defining a plurality of rungs of struts, and a plurality of spikes extending from at least some of the plurality of struts. The plurality of spikes are engaged with the valvular structure.

According to some aspects of the disclosure, there is provided a prosthetic valve comprising a main frame, an inner frame which is disposed radially inwardly from, and is coupled to, the main frame, and a valvular structure disposed between the main frame and the inner frame. The main frame comprises a plurality of struts defining a plurality of rows of cells, and is movable between a radially compressed state and a radially expanded state. The inner frame comprises a plurality of struts defining a plurality of rows of cells, and a plurality of spikes extending from at least some of the plurality of struts. The plurality of spikes are engaged with the valvular structure.

According to some aspects of the disclosure, there is provided a prosthetic valve comprising a main frame; an inner frame assembly comprising a plurality of sub-frame sections, each sub-frame section disposed radially inwardly from, and is coupled to, the main frame; and a valvular structure disposed between the main frame and the inner frame. The main frame comprises a plurality of struts, and is movable between a radially compressed state and a radially expanded state. Each sub-frame section comprises a plurality of struts and a plurality of spikes extending from at least some of the plurality of struts. The valvular structure comprises a movable portion and an engagement portion, wherein the movable portion is configured to regulate flow through the prosthetic valve. The plurality of spikes are engaged with the engagement portion of the valvular structure.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of one example of a prosthetic valve.

FIG. 1B is a perspective view of the prosthetic valve of FIG. 1A, without the outer skirt.

FIG. 1C is a perspective view of a main frame of the prosthetic valve of FIGS. 1A-1B.

FIG. 2 is a perspective view of an example of a leaflet structure that includes a thick sutured stitched along cusp edges of the leaflets.

FIG. 3 is an enlarged view of a portion of a prosthetic valve with the leaflet structure of FIG. 2 coupled to struts of its main frame.

FIG. 4 is a side elevation view of an example of a leaflet structure, shown in an unrolled or flattened configuration.

FIG. 5 is a side elevation view of an example of a single leaflet that can be used to form a leaflet structure, shown in a flattened configuration.

FIG. 6 is a perspective view of an example of an inner frame that can be positioned radially inward to a main frame of a prosthetic valve.

FIG. 7A is a side elevation view of the main frame of FIG. 1C, shown in a flattened configuration.

FIG. 7B is a side elevation view of the inner frame of FIG. 6, shown in a flattened configuration.

FIG. 8 is a perspective view from the inside of a portion of a prosthetic valve including a leaflet structure disposed between a main frame of the valve and an inner frame of the type shown in FIG. 6.

FIGS. 9A-9C are cross-sectional views taken along line 9-9 of FIG. 8, showing different examples of an inner frame equipped with spikes oriented at various angles.

FIG. 10 is a perspective view from the inside of a portion of a prosthetic valve including a leaflet structure disposed between a main frame of the valve and an inner frame equipped with laterally extendable struts.

FIG. 11 is a side elevation view of an example of a leaflet structure provided with apertures, shown in an unrolled or flattened configuration.

FIG. 12 is a perspective view of an example of a main frame provided with commissure support struts.

FIG. 13 is a perspective view from the inside of a portion of a prosthetic valve including a leaflet structure of the type shown in FIG. 11, disposed between a main frame of the type shown in FIG. 12, and an inner frame.

FIG. 14 is a side elevation view of an example of a single leaflet with a strip attached thereto, shown in a flattened configuration.

FIG. 15 is a side elevation view of another example of an inner frame, shown in a flattened configuration.

FIG. 16 is a perspective view from the inside of a portion of a prosthetic valve including a leaflet structure formed from leaflets of the type shown in FIG. 14, disposed between a main frame of the valve and an inner frame of the type shown in FIG. 15.

FIG. 17A is a side elevation view of an example of a leaflet structure provided with a skirt attached thereto, shown in an unrolled or flattened configuration.

FIG. 17B is a side elevation view of an example of a leaflet provided with a skirt attached thereto, shown in an unrolled or flattened configuration.

FIG. 18A is a partial cross-sectional exploded view of a portion of a prosthetic valve, showing a leaflet structure that includes a skirt, positioned between an inner frame and a main frame, prior to assembly of the three components.

FIG. 18B shows a partial cross-sectional view of the portion of the prosthetic valve from FIG. 18A, with the three components assembled together and the skirt folded over the main frame.

FIG. 19 is a side elevation view of an exemplary inner frame assembly that includes three sub-frame sections, shown in a flattened configuration.

FIG. 20 is a perspective view from the inside of a portion of a prosthetic valve including sub-frame sections of an inner frame assembly.

FIG. 21 shows an exemplary delivery assembly comprising a delivery apparatus carrying a prosthetic device.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

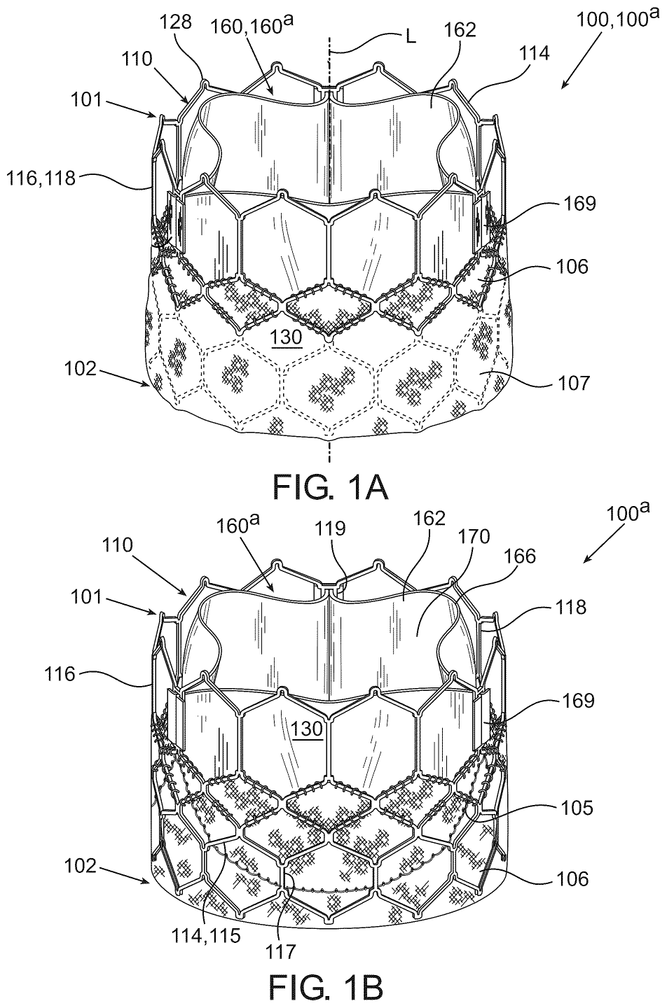

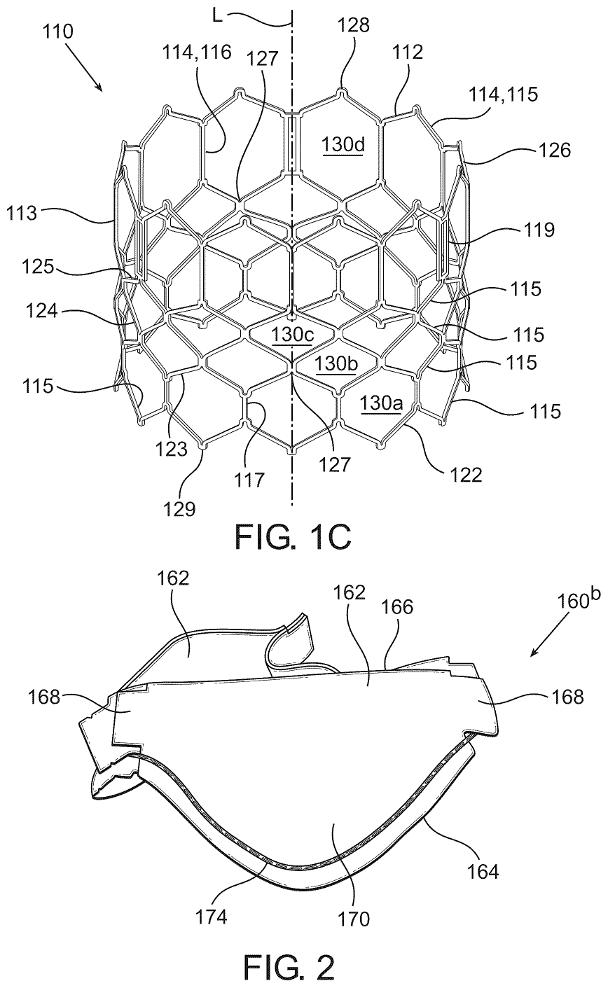

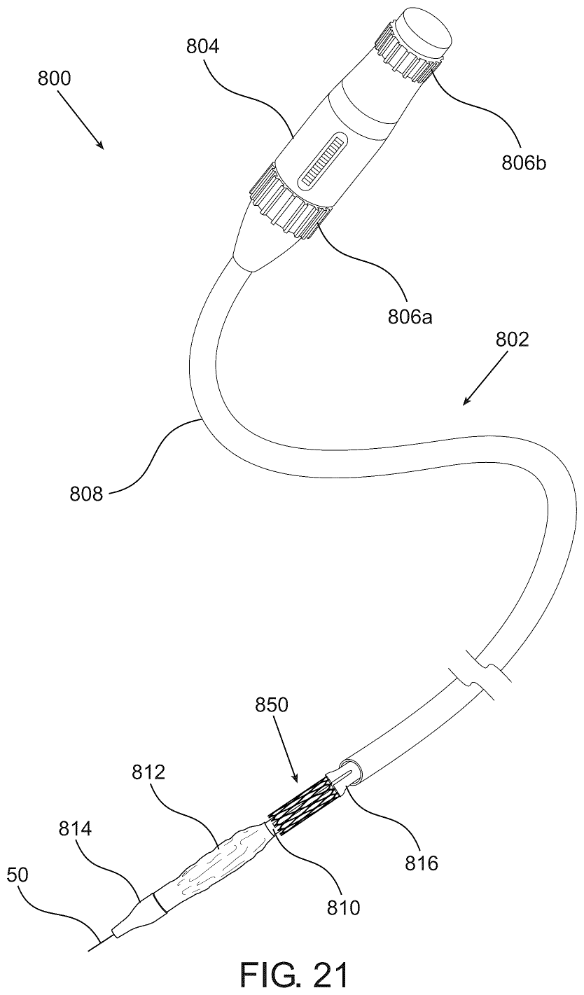

FIGS. 1A and 1B show perspective views of one example of a prosthetic valve 100, with and without an outer skirt 107 surrounding the main frame 110, respectively. FIG. 1C shows the main frame 110 without any other soft components attached thereto. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus (such as delivery apparatus 202 described in greater detail below with respect to FIG. 21) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100, 200, 300, 400, 500, 600, 700) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (as will be described in greater detail below with respect to FIG. 21). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term “plurality”, as used herein, means more than one.

FIGS. 1A-1B show an example of a prosthetic valve 100, which can be a balloon expandable valve, illustrated in an expanded state. The prosthetic valve 100 can comprise an outflow end 101 and an inflow end 102. In some instances, the outflow end 101 is the proximal end of the prosthetic valve 100, and the inflow end 102 is the distal end of the prosthetic valve 100. Alternatively, depending for example on the delivery approach of the valve, the outflow end can be the distal end of the prosthetic valve, and the inflow end can be the distal end of the proximal valve.

The term “proximal”, as used herein, generally refers to a position, direction, or portion of a device or a component of a device, which is closer to the user (for example, closer to an operator of a delivery apparatus utilized during an implantation procedure) and farther away from the implantation site.

The term “distal”, as used herein, generally refers to a position, direction, or portion of a device or a component of a device, which is farther away from the user and closer to the implantation site.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The valve 100 comprises an annular main frame 110 movable between a radially compressed configuration and a radially expanded configuration, and a valvular structure 160 mounted within the main frame 110. The main frame 110 can be made of various suitable materials, including plastically-deformable materials such as, but not limited to, stainless steel, a nickel based alloy (for example, a cobalt-chromium or a nickel-cobalt-chromium alloy such as MP35N alloy), polymers, or combinations thereof. When constructed of a plastically-deformable materials, the main frame 110 can be crimped to a radially compressed state on a balloon catheter, and then expanded inside a patient by an inflatable balloon or equivalent expansion mechanism. Alternatively or additionally, the main frame 110 can be made of shape-memory materials such as, but not limited to, nickel titanium alloy (for example, Nitinol).

When constructed of a shape-memory material, the main frame 110 can be crimped to a radially compressed state and restrained in the compressed state by insertion into a shaft or equivalent mechanism of a delivery apparatus.

In the example illustrated in FIGS. 1A-1C, the main frame 110 is an annular, stent-like structure comprising a plurality of intersecting struts 114. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 114 may be any elongated member or portion of the main frame 110. The main frame 110 can include a plurality of strut rungs that can collectively define one or more rows of cells 130. The main frame 110 can have a cylindrical or substantially cylindrical shape having a constant diameter from the inflow end 102 to the outflow end 101 as shown, or the frame can vary in diameter along the height of the frame, as disclosed in U.S. Pat. No. 9,155,619, which is incorporated herein by reference.

The end portions of the struts 114 are forming apices 128 at the outflow end 101 and apices 129 at the inflow end 102. The struts 114 can intersect at additional junctions 127 formed between the outflow apices 128 and the inflow apices 129. The junctions 127 can be equally or unequally spaced apart from each other, and/or from the apices 128, 129, between the outflow end 101 and the inflow end 102.

The struts 114 can include a plurality of angled struts 115 and vertical or axial struts 116. FIGS. 1A-1C show an exemplary prosthetic valve 100 that can be representative of, but is not limited to, a balloon expandable prosthetic valve. The main frame 110 of the prosthetic valve 100 illustrated in FIG. 1C comprises rungs of angled struts 122, 123, 124, 125, 126, and axial struts 117, 118 disposed between some of the rungs of angled struts. In such implementations of the frame, the struts can be pivotable or bendable relative to each other, so as to permit frame expansion or compression. For example, the main frame 110 can be formed from a single piece of material, such as a metal tube, via various processes such as, but not limited to, laser cutting, electroforming, and/or physical vapor deposition, while retaining the ability to collapse/expand radially in the absence of hinges and like.

A conventional valvular structure 160, shown also for example in FIG. 2, can include a plurality of leaflets 162 (for example, three leaflets), positioned at least partially within the main frame 110, and configured to regulate flow of blood through the prosthetic valve 100 from the inflow end 102 to the outflow end 101. While three leaflets 162 arranged to collapse in a tricuspid arrangement, are shown in the example illustrated in FIGS. 1A-1B and 2, it will be clear that a prosthetic valve 100 can include any other number of leaflets 162. Adjacent leaflets 162 can be arranged together to form commissures 169 that are coupled (directly or indirectly) to respective portions of the main frame 110, thereby securing at least a portion of the valvular structure 160 to the main frame 110. The leaflets 162 can be made from, in whole or part, biological material (for example, pericardium), bio-compatible synthetic materials, or other such materials. Further details regarding transcatheter prosthetic heart valves, including the manner in which the valvular structures 160 can be coupled to the main frame 110 of the prosthetic valve 100, can be found, for example, in U.S. Pat. Nos. 6,730,118, 7,393,360, 7,510,575, 7,993,394, and 8,652,202, and U.S. Patent Application Publication No. 2018/0325665, all of which are incorporated herein by reference in their entireties.

As shown for example in FIG. 2, three separate leaflets 162 can collectively define the valvular structure 160 in some cases. Each conventional separate leaflet 162 can have a rounded cusp edge 164 opposite a free edge 166, and a pair of generally oppositely-directed tabs 168 separating the cusp edge 164 and the free edge 166. The cusp edge 164 in such cases forms a single scallop. Each separate leaflet 162 further comprises an inner surface (not annotated), defined as the surface facing the valve central longitudinal axis L, and an outer surface (not annotated), opposite thereto so as to face the main frame 110.

When such leaflets 162 are coupled to the main frame and to each other, the lower edge of the resulting valvular structure 160 desirably has an undulating, curved scalloped shape. By forming the leaflets with this scalloped geometry, stresses on the leaflets 162 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

The leaflets 162 define a non-planar coaptation plane (not annotated) when their free edges 166 co-apt with each other to seal blood flow through the prosthetic valve 100. Leaflets 162 can be secured to one another at their tabs 168 to form commissures 169 of the valvular structure 160, which can be secured, directly or indirectly, to structural elements connected to the main frame 110 or integrally formed as portions thereof, such as commissure posts, commissure windows, and the like. When secured to two other leaflets 162 to form valvular structure 160, the cusp edges 164 of the leaflets 162 collectively form the scalloped line 105 of the valvular structure 160. Each leaflet 162 comprises a leaflet body 170, defined between the line of attachment of the leaflet to the main frame, for example along scalloped line 105, and the free edge 166. The leaflet body 170 defines the movable portion of the leaflet 162, free to move toward the main frame 110 in an open state of the valvular structure 160, and toward central longitudinal axis L to co-apt with other leaflets 162 in a closed state of the valvular structure 160.

In examples, the prosthetic valve 100 can further comprise at least one skirt or sealing member. FIGS. 1A-1B show an example of a prosthetic valve 100^{a} that includes an inner skirt 106, which can be secured to the inner surface 112 of the main frame 110. Such an inner skirt 106 can be configured to function, for example, as a sealing member to prevent or decrease perivalvular leakage. An inner skirt 106 can further function as an anchoring region for valvular structure 160a to the main frame 110, and/or function to protect the leaflets 162 against damage which may be caused by contact with the main frame 110, for example during valve crimping or during working cycles of the prosthetic valve 100. FIG. 1B shows an inner skirt 106 disposed around and attached to the inner surface 112 of main frame 110, wherein the valvular structure 160^{a} is sutured to the inner skirt 106 along scalloped line 105. Additionally, or alternatively, the prosthetic valve 100 can comprise an outer skirt 107 mounted on the outer surface 113 of main frame 110, configure to function, for example, as a sealing member retained between the main frame 110 and the surrounding tissue of the native annulus against which the prosthetic valve is mounted, thereby reducing risk of paravalvular leakage (PVL) past the prosthetic valve 100.

Any of the inner skirt 106 and/or outer skirt 107 can be made of various suitable biocompatible materials, such as, but not limited to, various synthetic materials (for example, PET) or natural tissue (for example pericardial tissue). In some cases, the inner skirt 106 can be formed of a single sheet of material that extends continuously around the inner surface 112 of main frame 110. In some cases, the outer skirt 107 can be formed of a single sheet of material that extends continuously around the outer surface 113 of main frame 110.

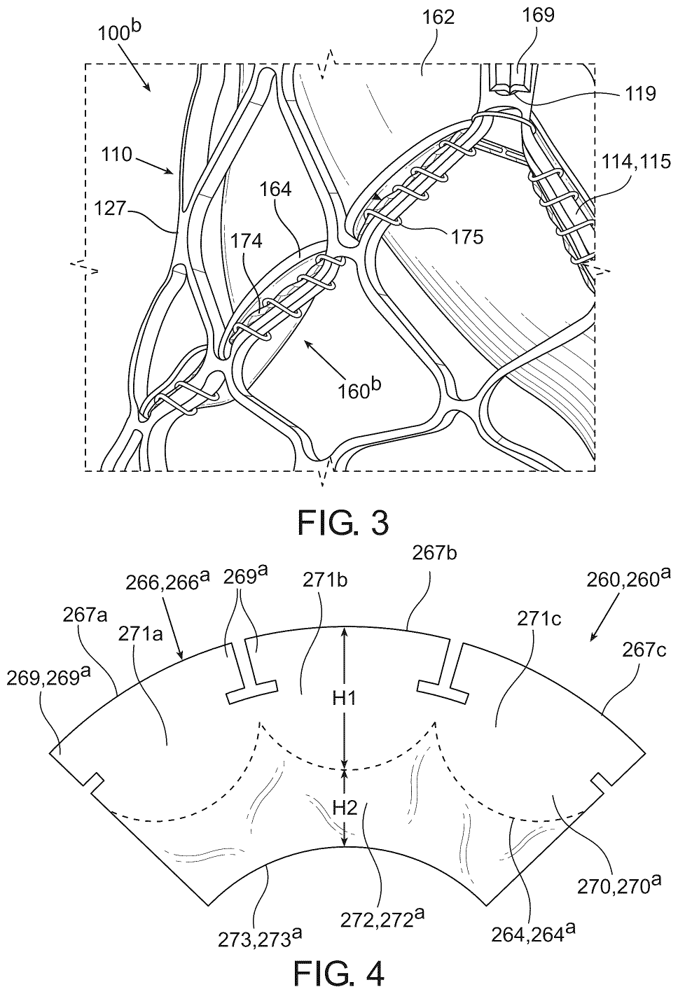

FIG. 3 shows a zoomed in view of another attachment configuration of a valvular structure 160^{b}, shown in FIG. 2, to a main frame 110 of a prosthetic valve 100^{b}, in which the leaflet end portions adjacent cusp edges 164, are sutured to struts 114 that can generally follow the contour of the cusp edges 164, in a manner that can allow for the elimination of an inner skirt in the assembled prosthetic valve. Reducing the number of soft components of the prosthetic valve (for example, by eliminating the inner skirt and suturing the leaflets directly to the frame) can simplify a process for assembling the prosthetic valve. For example, assembling prosthetic valves including an inner skirt can result in a prolonged assembly time which involves suturing each of the leaflets to the inner skirt and then suturing the inner skirt to the frame of the prosthetic valve.

FIG. 2 shows separate leaflets 162 secured together into an exemplary valvular structure 160^{b} with a thick suture line 174 disposed adjacent cusp edge 164 and tracking the contour of cusp edge 164 of each of the leaflets 162. In such examples, a connecting suture line 174 can enable securing of the valvular structure 160^{b} directly to a main frame 110, without having to extend sutures through the leaflet during assembly to the frame. As shown in FIG. 3, direct attachment of the valvular structure 160 of FIG. 2 to the main frame 110 can be achieved by looping a connecting suture 175 around the struts 114 and through or around portions of the thick suture 174.

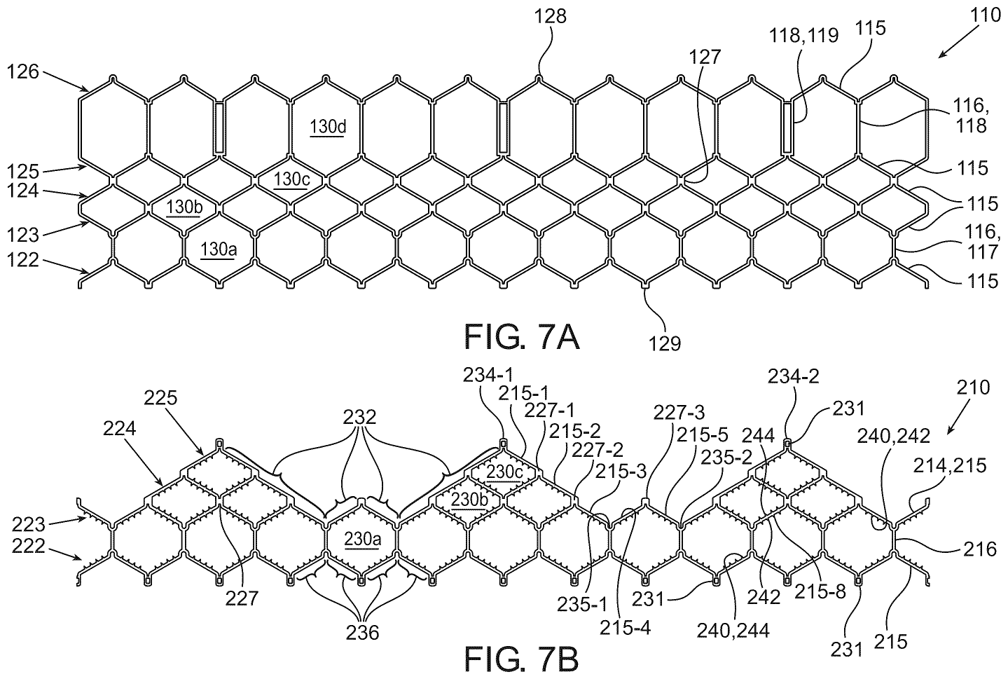

FIGS. 1C and 7A show the main frame 110 of the prosthetic valve 100 with the other components, such as leaflets and skirts, removed. While FIG. 1C shows the main frame 110 in an annular configuration, corresponding to its functional configuration, FIG. 7A shows the main frame 110 in a flat configuration for purposes of illustration. The main frame 110 can comprise, in examples, a plurality of rows or rungs of angled struts, as well as axial struts that can extend between some rungs of angled struts.

In the example illustrated in FIGS. 1C and 7A, the main frame 110 comprises a first rung 122 of circumferentially extending angled struts 115 that intersect at inflow apices 129 along the inflow end 102, a second rung 123 of circumferentially extending angled struts 115, a third rung 124 of circumferentially extending angled struts 115, a fourth rung 125 of circumferentially extending angled struts 115, and a fifth rung 126 of circumferentially extending angled struts 115 that intersect at outflow apices 128 along the outflow end 101.

A plurality of substantially straight distal axial struts 117 can be used to interconnect the angled struts of first rung 122 with the angled struts of the second rung 123. Specifically, each distal axial strut 117 extends from a location defined by the convergence of upper ends of two angled struts of the first rung 122 to another location defined by the convergence of lower ends of two angled struts of the second rung 123.

Similarly, a plurality of substantially straight proximal axial struts 118 can be used to interconnect the angled struts of fifth rung 126 with the angled struts of the fourth rung 125. Specifically, each proximal axial strut 118 extends from a location defined by the convergence of upper ends of two angled struts of the fourth rung 125 to another location defined by the convergence of lower ends of two angled struts of the fifth rung 126.

The axial length of the proximal axial struts 118 can be different than that of the distal axial struts 117. For example, in the illustrated configuration the proximal axial struts 118 can be longer than the distal axial struts 117. In examples, at least some (for example, three) of the proximal axial struts 118 can define axially extending window frame portions, also termed commissure windows 119, configured to mount respective commissures 169 of the valvular structure 160.

The struts 114 collectively define a plurality of cells 130 of the main frame 110. At the inflow end of the main frame 110, angled struts of the first rung 122, distal axial struts 117, and angled struts of the second rung 123, define a first or lowermost row of cells 130a. The angled struts of the second rung 123 and the third rung 124 define a second row of cells 130b. The angled struts of the third rung 124 and the fourth rung 125 define a third row of cells 130c. The angled struts of the fourth rung 125, proximal axial struts 118, and angled struts of the fifth rung 126, define a fourth or uppermost row of cells 130d.

FIG. 21 illustrates exemplary delivery assembly 800 that includes a delivery apparatus 802 adapted to deliver a balloon expandable prosthetic device 850, which can be any prosthetic valve of the current disclosure (for example, prosthetic valve 100, 200, 300, 400, 500, 600, 700). According to some examples, the delivery apparatus 802 includes a handle 804 and at least one catheter extending therefrom, configured to carry a prosthetic device 850 (for example, any of prosthetic valve 100, 200, 300, 400, 500, 600, 700) in a crimped state through the patient's vasculature. An exemplary delivery assembly 800 comprises an exemplary delivery apparatus 802 configured to carry a balloon expandable prosthetic valve. The delivery apparatus 802 can comprise a balloon catheter 810 having an inflatable balloon 812 mounted on its distal end. A balloon expandable prosthetic valve (for example, any of prosthetic valve 100, 200, 300, 400, 500, 600, 700) can be carried in a crimped state over the balloon catheter 810.

In some examples, a delivery apparatus 802 further comprises an outer delivery shaft 808. Optionally, an outer delivery shaft 808 of a delivery apparatus 802 can concentrically extend over the balloon catheter 810. In some examples, delivery apparatus 802 can further comprise a push shaft 816 disposed over the balloon catheter 810, optionally between the balloon catheter 810 and the outer delivery shaft 808.

The outer delivery shaft 808, the push shaft 816, and the balloon catheter 810, can be configured to be axially movable relative to each other. For example, a proximally oriented movement of the outer delivery shaft 808 relative to the balloon catheter 810, or a distally oriented movement of the balloon catheter 810 relative to the outer delivery shaft 808, can expose the prosthetic device 850 from the outer delivery shaft 808.

A delivery apparatus 802 can further include a nosecone 814 carried by a nosecone shaft (hidden from view in FIG. 21). The nosecone shaft can extend through a lumen of the balloon catheter 810. The nosecone shaft and the nosecone 814 can be designed to allow passage of a guidewire 50 therethrough, such that the delivery assembly 200 can be advanced inside a patient's body over guidewire 50.

The proximal ends of the balloon catheter 810, the outer delivery shaft 808, the push shaft 816, and/or the nosecone shaft, can be coupled to the handle 804. During delivery of the prosthetic device 850, the handle 804 can be maneuvered by an operator (for example, a clinician or a surgeon) to axially advance or retract components of the delivery apparatus 802, such as the nosecone shaft, the outer delivery shaft 808, the balloon catheter 810 and/or the push shaft 816, through the patient's vasculature and/or along the target site of implantation, as well as to inflate the balloon 812 mounted on the balloon catheter 810, so as to expand the prosthetic device 850, and to deflate the balloon 812 and retract the delivery apparatus 802 once the prosthetic device 850 is mounted in the implantation site.

The handle 804 can include a steering mechanism configured to adjust the curvature of the distal end portion of the delivery apparatus 802. In the illustrated example, the handle 804 includes an adjustment member, such as the illustrated rotatable knob 806a, which in turn is operatively coupled to the proximal end portion of a pull wire (not shown). The pull wire can extend distally from the handle 804 through the outer delivery shaft 808 and has a distal end portion affixed to the outer delivery shaft 808 at or near the distal end of the outer delivery shaft 808. Rotating the knob 806a can increase or decrease the tension in the pull wire, thereby adjusting the curvature of the distal end portion of the delivery apparatus 802. Further details on steering or flex mechanisms for the delivery apparatus can be found in U.S. Pat. No. 9,339,384, which is incorporated by reference herein. The handle 804 can further include an adjustment mechanism including an adjustment member, such as the illustrated rotatable knob 806b. The adjustment mechanism can be configured to adjust the axial position of the push shaft 816 relative to the balloon catheter 810. The handle can include additional knobs to control additional components of the delivery apparatus 802, such as positioning members that will be described in greater detail below.

The prosthetic device 850 can be carried by the delivery apparatus 802 during delivery in a crimped state, and expanded, for example by balloon inflation, to secure it in a native heart valve annulus (such as an aortic annulus) or against a previously implanted prosthetic valve (for example, during valve-in-valve implantation procedures). The prosthetic device 850 can be initially crimped over the balloon catheter 810, proximal to the balloon 812. Because prosthetic device 850 is crimped at a location different from the location of balloon 812, prosthetic device 850 can be crimped to a lower profile than would be possible if it was crimped on top of balloon 812. This lower profile permits the clinician to more easily navigate the delivery assembly 800 (including crimped prosthetic device 850) through a patient's vasculature to the treatment location. The lower profile of the crimped prosthetic valve is particularly helpful when navigating through portions of the patient's vasculature which are particularly narrow, such as the iliac artery.

The balloon 812 can be secured to balloon catheter 810 at the balloon's proximal end, and to either the balloon catheter 810, the nosecone shaft, or the nosecone 814 at its distal end. The distal end portion of the push shaft 816 is positioned proximal to the outflow end of prosthetic device 850 (for example, outflow end 101 in the case of prosthetic valve 100).

When reaching the site of implantation, the deflated balloon 812, carrying crimped prosthetic device 850 thereover, can be advanced to the target site to expand the prosthetic valve. Prior to balloon 812 inflation, the push shaft 816 is advanced distally, allowing its distal end portion to contact and push against the outflow end of prosthetic device 850, pushing the device 850 distally therewith. The distal end of push shaft 816 is dimensioned to engage with the outflow end of prosthetic device 850 in a crimped configuration of the device. In some examples, the distal end portion of the push shaft 816 can be flared radially outward, to terminate at a wider-diameter that can contact the prosthetic device 850 in its crimped state. Push shaft 816 can then be advanced distally, pushing the prosthetic device 850 therewith, until the crimped prosthetic device 850 is disposed around the balloon 812, at which point the balloon 812 can be inflated to radially expand the prosthetic device 850. Once the prosthetic device 850 is expanded to its functional diameter within a native annulus or within a previously implanted prosthetic valve, the balloon 812 can be deflated, and the delivery apparatus 802 can be retrieved from the patient's body.

In some examples, any exemplary delivery assembly and/or prosthetic valve of the current disclosure can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Conventional prosthetic valves, such as prosthetic valve 100, include a valvular structure 160 with the leaflets 162 that can be either sutured to an inner skirt 106, as illustrated and described for a prosthetic valve 100^{a}, or sutured directly to struts 114 of the main frame 110, as illustrated and described for a prosthetic valve 100^{b}. The suturing process tends to be labor-intensive, and the quality of the result may depend on the skill-level of the assembler. Moreover, the tension applied to the suture may not be well controlled, which can effect local geometries of the leaflets 162. All of these factors may adversely impact the functionality and structural integrity of the valvular structure 160 in the long term. Described below are prosthetic valve that can include an inner frame disposed radially inwardly from, and is coupled to, the main frame, with valvular structures disposed between the main frame and the inner frame, secured in position by a plurality of spikes extending from struts of the inner frame, thereby obviating the use of sutures stitched through the leaflet material.

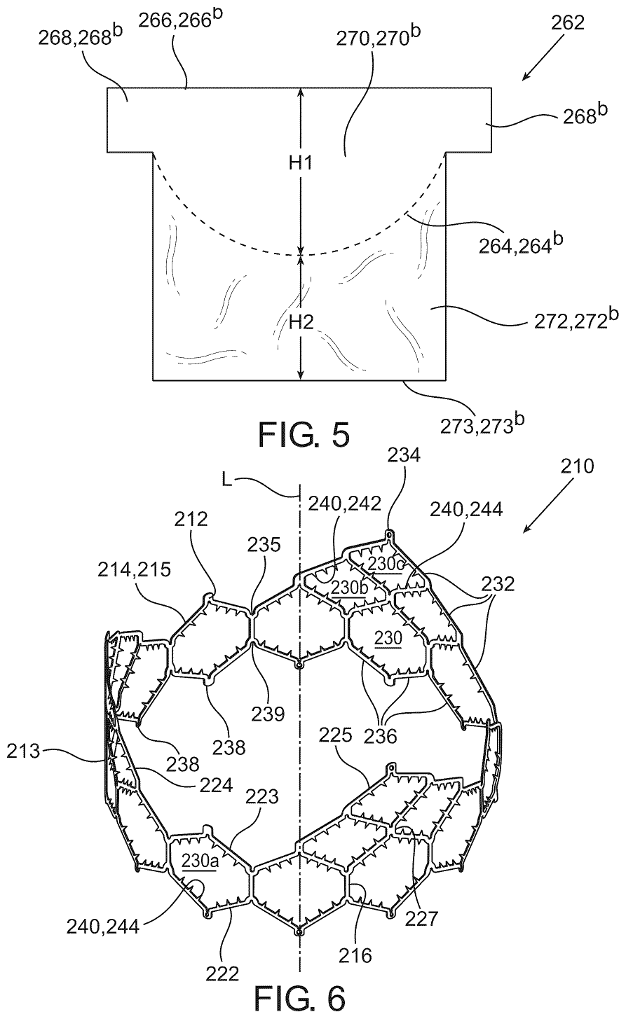

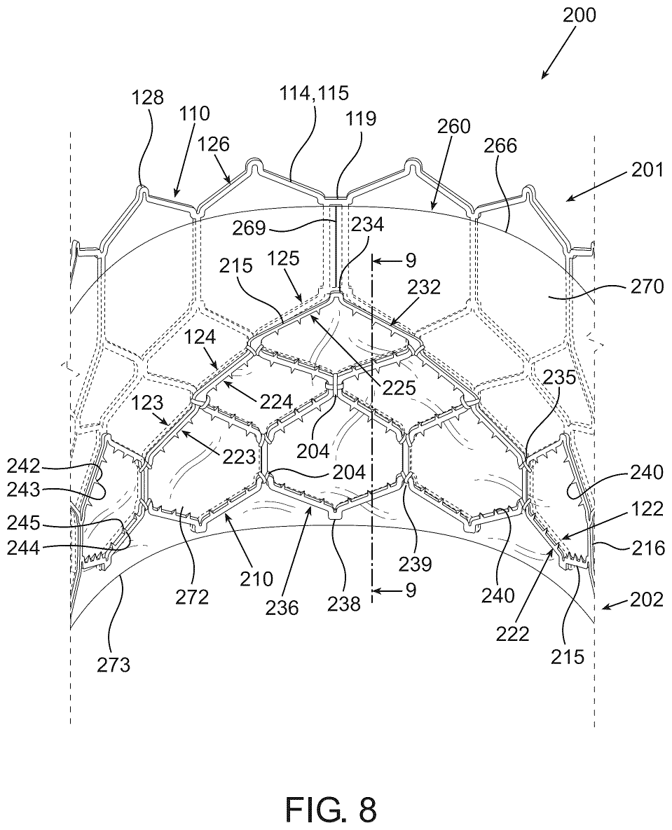

Referring to FIGS. 4-6 and 7B-8, a prosthetic valve 200 (shown, for example, in FIG. 8) can include a valvular structure 260 (examples of which are shown in FIGS. 4 and 5) disposed between a main frame 110, and an inner frame 210 (shown, for example, in FIGS. 6 and 7B) which is disposed radially inwardly from, and is coupled to, the main frame 110. The prosthetic valve 200 can comprise an outflow end 201 and an inflow end 202, and can be in some examples, as described for prosthetic valve 100, a balloon expandable valve, though it is to be understood that other valve types are similarly contemplated. The inner frame 210 comprises leaflet engaging features, such as spikes, designed to engage with the valvular structure 260, for example along an engagement portion 272 thereof, in a manner that reliably retains the engagement portion 272 attached and immovable between the frames 110, 210.

Valvular structure 260 includes a movable portion 270 extending distally from a free edge 266 thereof, and an engagement portion 272 extending proximally from an engagement portion distal end 273 opposite to the free edge 266. The engagement portion 272 is the portion of the valvular structure 260 configured to reside between the inner frame 210 and main frame 110 when the prosthetic valve 200 is assembled. A proximal attachment line 264 designates the border between the engagement portion 272 and the movable portion 270 of the valvular structure. The movable portion 270 is defined between the free edge 266 and the engagement portion 272, for example between the free edge 266 and a proximal attachment line 264. The movable portion 270 defines the portion of the valvular structure 260 which is not engaged by the inner frame 210 (i.e., disposed above the inner frame 210) when the prosthetic valve 200 is assembled, thus allowed to freely move toward the main frame 110 in an open state of the valvular structure 260, and toward central longitudinal axis L in a closed state of the valvular structure 260.

In some examples, movable portion 270 and engagement portion 272 of a valvular structure 260 constitute different regions of a unitary continuous piece of material. Such a unitary continuous piece of material can be homogenous, having identical material properties, including material rigidity or stiffness, along both movable portion 270 and engagement portion 272. In such cases, proximal attachment line 264 may be defined primarily by the upper or proximal edge of the inner frame 210 when prosthetic valve 200 is assembled, such that the portion of the valvular structure 260 distal to the proximal edge of inner frame 210 is defined as the engagement portion 272, and the portion of the valvular structure 260 proximal to the proximal edge of inner frame 210 is defined as the movable portion 270.

In some examples, the proximal attachment line 264 comprises a visual marking, such as a visible die-colored marking, a suture extending along the attachment line 264, and the like, which can help during the assembly procedure to properly align the valvular structure 260 between the frames 110, 210.

In some examples, the material properties of valvular structure 260 are not homogenous. Specifically, engagement portion 272 can be stiffer, in some examples, than movable portion 270. A unitary piece of material can be provided with non-homogenous material properties. For example, valvular structure 260 can be formed from natural tissue, such as pericardial tissue, undergoing biological treatment procedures that can include subjecting the tissue to cross-linking agents, which can influence its final material properties. In such an example, each of the movable portion 270 and engagement portion 272 can be subjected to a different biological procedure, including being subjected to different cross-linking agents, subjected to such agents for different time durations, or other procedural variations, adapted to result in an engagement portions that can be stiffer than the corresponding movable portion 270.

The movable portion 270 can be designed to have material properties substantially similar to those of conventional leaflets 162 described above with respect to FIGS. 1A-3, allowing this portion to move between the closed and open states in a similar manner. The engagement portion 272 can be stiffer compared to the movable portion 270, facilitating easier penetration of spikes 240 of the inner frame 210 thereinto, and improving engagement retention with the spikes. The engagement portion 272 can be semi-rigid, meaning that it is stiffer or more rigid than the movable portion 270, yet flexible enough to transition between the crimped and expanded configurations of prosthetic valve 200 without experiencing material failure and without resisting such transitions of the valve.

In some examples, the valvular structure 260 can be provided as a unitary continuous material, with the engagement portion 272 reinforced by coating with a reinforcing layer, or otherwise attached along a surface thereof to a reinforcing layer that imparts additional stiffness thereto, such as a polymeric layer or any other suitable layer. The stiffness of any portion of a valvular structure can be measured by following the general procedures set forth in ASTM D790.

In some examples, the valvular structure 260 can be formed from two different materials, joined to each other along proximal attachment line 264 (for example, by suturing, gluing, welding, and the like), wherein the material forming the engagement portion 272 can be stiffer than the material forming the movable portion 270.

FIG. 4 shows one example of a valvular structure 260^{a}, that can be formed of a unitary piece of material designed to cover the entire circumference of prosthetic valve 200 between frames 210 and 110. For example, movable portion 270^{a} can define a plurality of movable regions 271, such as movable regions 271a, 271b and 271c shown in the illustrated configuration. The valvular structure 260^{a} can be cut along an upper region thereof in a manner that separates between adjacent tabs 268^{a}, in which case the free edge 266^{a} can include a corresponding plurality of free edge portions 267, such as three edge portions 267a, 267b and 267c that can be separate from each other, as shown in the illustrated example.

In some examples, a valvular structure 260^{a} can include a unitary movable portion 270^{a} defining a plurality of movable regions (for example, regions 271a, 271b, 271c), while the engagement portion 272^{a} can be made of a different (for example, stiffer) material joined to the movable portion 270^{a} along proximal attachment line 264^{a}, or can be made from the same material, continuous with movable portion 270^{a}, either having homogenous material properties, or provided with different material properties—such as by being subjected to a different biological treatment procedure, being coated or attached to another layer, and the like.

Since each two adjacent tabs 268^{a} are approximated to each other and joined to form a commissure 269^{a} that can extend radially outward when assembled in the valve 200, the valvular structure 260^{a} can, in examples, follow a non-linear but rather arcuate profile in a flattened configuration, as shown in FIG. 4, such that both the free edge 266 and the engagement portion distal end 273 follow an arcuate path, allowing the valvular structure 260^{a} to assume a final substantially circular configuration when mounted between the frames 110, 210.

Another example of a valvular structure 260^{b} can be formed from separate leaflets 262 of the type shown in FIG. 5. Each leaflet 262 can include a movable portion 270^{b} extending distally from a free edge 266^{b}, with two opposing tabs 268^{b}. The engagement portion 272^{b} can be made of a different (for example, stiffer) material joined to the movable portion 270^{b} along proximal attachment line 264^{b}, or both can be made from the same material, either having homogenous material properties, or engagement portion 272^{b} provided with different material properties—such as by being subjected to a different biological treatment procedure, being coated or attached to another layer, and the like.

In examples, the proximal attachment line 264 is non-linear. For example, proximal attachment line 264^{b} defined for a single leaflet 262 or along one movable region 271 of valvular structure 260^{a}, can generally track a shape similar to an arcuate cusp edge 164 shown for conventional leaflets 162, such that the shape of proximal attachment line 264 formed by combining three leaflets 262 joined together to form valvular structure 260^{b}, or its shape along the entire circumference of valvular structure 260^{a}, can generally resemble a scalloped line shape, as illustrated. In some cases, the proximal attachment line 264 is dictated by the shape of the upper edge of inner frame 210, and thus can follow other patterns, such as substantially zig-zagged paths and the like, that can have undulating-like shapes with portions extending in proximal and distal directions, forming a plurality of peaks and throughs along the proximal attachment line 264.

Movable portion 270 can be dimensioned to generally mimic the shape and size of leaflet body 170 of a conventional leaflet 162 in the case of valvular structure 260^{b}, or a combination of leaflet bodies 170 of a conventional valvular structure 160 in the case of valvular structure 260^{a}, of the types described above with respect to FIGS. 1A-3. For example, a maximal movable portion height H1 can be defined between a middle point along the free edge 266 (including free edge 2661 or free edge portion 267) and an opposite lowermost point along the proximal attachment line 264, such as a middle point along proximal attachment line 264. Movable portion height H1 can be designed to be similar to an equivalent height of the leaflet body 170 of a conventional leaflet 162, for example measured between a middle point of the free edge 166 and an opposing middle point of the cusp edge 164.

A minimal engagement portion height H2 can be defined between the lowermost point along the proximal attachment line 264, such as a middle point along proximal attachment line 264, and a vertically opposing point at the engagement portion distal end, as illustrated in FIGS. 4 and 5, such that the combination of H1 and H2 results in a total height of the valvular structure 260 (as well as the height of any single leaflet 262). Thus, the total height of valvular structure 260 and/or leaflet 262 is necessarily greater than an equivalent conventional valvular structure 160 and/or leaflet 162 of the types described above with respect to FIGS. 1A-3.

The inner frame 210 comprises at least one rung of struts equipped with spikes 240 configured to penetrate into valvular structure 260 and retain engagement therewith. The spikes 240 can be oriented in the proximal and/or distal directions, and can extend radially outward (toward main frame 110), but not radially inward (i.e., not toward central axis L). The inner frame 210 comprises a plurality of struts 214 configured in an annular shape. Inner frame 210 further defines an inner surface 212 facing central axis L and an outer surface 213 facing the main frame 110, wherein surfaces 212, 213 are defined by the struts 214 without the spikes.

FIGS. 6 and 7B show one example of an inner frame 210. FIG. 6 shows the inner frame 210 in an annular configuration, detached from the prosthetic valve 200, and FIG. 7B shows the inner frame 210 in a flat configuration for purposes of illustration. The inner frame 210 can comprise, in examples, a plurality of rows or rungs of angled struts, as well as axial struts that can extend between some rungs of angled struts. In the example illustrated in FIGS. 6 and 7B, the inner frame 210 comprises a first rung 222 of circumferentially extending angled struts 215, a second rung 223 of circumferentially extending angled struts 215, a third rung 224 of circumferentially extending angled struts 215, and a fourth rung 225 of circumferentially extending angled struts 215.

A plurality of substantially straight axial struts 216 can be used to interconnect the angled struts of first rung 222 with the angled struts of the second rung 223. Specifically, each axial strut 216 extends from a location defined by the convergence of upper ends of two angled struts of the first rung 222 to another location defined by the convergence of lower ends of two angled struts of the second rung 223.

The struts 214 collectively define a plurality of cells 230 of the inner frame 210. Angled struts of the first rung 222, axial struts 216, and angled struts of the second rung 223, define a first or lowermost row of cells 230a. The angled struts of the second rung 223 and the third rung 224 define a second row of cells 230b. The angled struts of the third rung 224 and the fourth rung 225 define a third or uppermost row of cells 230c.

Inner frame 210 extends between an outflow border 232, defined by the proximal-most angled struts 215 along the circumference of the inner frame 210, and an opposite inflow border 236, defined by the distal-most angled struts 215 along the circumference of the inner frame 210. The outflow border 232 constitutes the upper edge of inner frame 210, which is aligned with, and in some cases even defines, the proximal attachment line 264 of valvular structure 260.

As shown, one or more rungs of angles struts 215 can be continuous, and one or more other rungs of angled struts 215 of inner frame 210 can be discontinuous. A discontinuous rung of struts is defined as a rung in which at least two struts are spaced from each other without being interconnected by any other strut disposed therebetween along the circumference of the same rung. Similarly, one or more rows of cells 230 can be continuous, and one or more rows of cells 230 of inner frame 210 can be discontinuous. A discontinuous row of cells is defined as a row in which at least two closed cells are spaced from each other without being interconnected by any other closed cell disposed therebetween along the circumference of the same row of cells.

In examples, at least one rung of angled struts 215 of inner frame 210 is continuous, and at least one other rung of angled struts 215 is discontinuous. In examples, at least two rung of angled struts 215 of inner frame 210 are continuous, and at least one other rung of angled struts 215 is discontinuous. Two continuous rungs of angled struts are required to define one continuous row of cells 230. In examples, at least one row of cells 230 of inner frame 210 is continuous, and at least one other row of cells 230 is discontinuous.

In the illustrated example, first rung 222 and second rung 223 are continuous, together defining a continuous row of cells 230a, while third rung 224 and fourth rung 225 are discontinuous, resulting in discontinuous rows of cells 230b and 230c. Moreover, an inner frame 210 that includes more than one discontinuous rung of angled struts, and more than one discontinuous row of cells, can have varying number of angled struts 215 in any of the rungs and varying number of closed cells 230 in any of the rows. When two or more discontinuous rungs of angled struts 215 are provided, proximal rung or rungs having fewer angled struts 215 than distal rung or rungs, will result in a generally undulating or scallop-like shaped outflow border 232. Similarly, when two or more discontinuous rows of cells 230 are provided, proximal row or rows having fewer cells 230 than distal row or rows of cells, will result in a generally undulating or scallop-like shaped outflow border 232.

In the illustrated example, first rung 222 and second rung 223 are continuous rungs, together defining a lowermost continuous row of cells 230a, wherein the angled struts 215 of first rung 222 define the inflow border 236, shown to have a generally zig-zagged pattern with a series of inflow border peak junctions 238, which are the lowermost or distal-most junctions 227 of the inner frame 210, and inflow border through junctions 239 which are the proximal-most junctions 227 along inflow border 236, proximal to inflow border peak junctions 238. The inflow border peak junctions 238 and inflow border through junctions 239 are all defined by intersecting angled struts 215 of a single rung, which is the first rung 222 in the illustrated example.

In the illustrated example, third rung 224 and fourth rung 225 are discontinuous rungs, with fourth rung 225 having fewer angled struts 215 than third rung 224, resulting in discontinuous second row of cells 230b and discontinuous third row of cells 230c, with the third or uppermost row having fewer cells 230c than the second row of cells 230b. FIG. 7B illustrates an example of the first and second rungs 222, 223 including 24 struts 214, each, the third rung 224 comprising 12 struts 214, and the fourth rung 225 comprising 6 struts 214. This results in all angled struts 215 of the fourth rung 225, and selected angled struts 215 of the third rung 224 and the second rung 223, defining together the outflow border 232 following along a generally undulating or scallop-like path, defining outflow border peak junctions 234 which are the uppermost or proximal-most junctions 227 of the inner frame 210, and outflow border through junctions 235 which are the distal-most junctions 227 along outflow border 232, distal to outflow border peak junctions 234.

For example, two successive outflow border peak junctions 234-1 and 234-2 are indicated in FIG. 7B, with two consecutive outflow border through junctions 235-1 and 235-2 disposed therebetween. A portion of the outflow border 232 downwardly extending from outflow border peak junction 234-1 to outflow border through junction 235-1 includes an angled strut 215-1 of fourth rung 225 extending from outflow border through junction 235-1 to junction 227-1; an angled strut 215-2 of third rung 224 extending from junction 227-1 to junction 227-2; and an angled strut 215-3 of second rung 223 extending from junction 227-2 to outflow border through junction 235-1. A bottom portion of the outflow border 232 is defined by consecutive angled struts 215 of the second rung 223, such as angled strut 215-3, followed by angled strut 215-4 extending from outflow border through junction 235-1 to junction 227-3, a subsequent angled strut 215-5 extending from junction 227-3 to outflow border through junction 235-2, and so on.

The inner frame 210 can include, as in the illustrated example, a plurality of rows of cells, with a lowermost row of cells 230a defining the inflow border peak junctions 238, and an uppermost row of cells 230c defining the outflow border peak junctions 234. In examples, at least one row of cells includes more cells than another row of cells of the inner frame 210. In the illustrated example, the lowermost row of cells 230a is a continuous row of cells, such that each cell 230a is directly attached to adjacent cells 230a on both of its sides. A direct connection between adjacent cells in the same row of cells, refers to both cells sharing at least one mutual junction 227. In the illustrated example, each cells 230a shared a common axial strut 216 defining two mutual junctions 227 with another cell 230a on each side.

As further shown in the illustrated example, the second row of cells 230b is a discontinuous row, wherein each cells 230b is directly attached to an adjacent cells 230b on one side thereof, and is not directly connected to any other cells 230b of the same row on the opposite side. Thus, while the lowermost row of cells includes 12 cells 230a in the illustrated example, the second row of cells includes 6 cells 230b. The uppermost row of cells 230c is also a discontinuous row, wherein each of the cells 230c is not directly connected to any other cells 230c of the same row, resulting in a total of 3 cells 230c in the illustrated example.

In the illustrated example, the inflow border 236 is defined solely by struts 214 of the lowermost row of cells 230a, and more precisely, solely by angled struts 215 of the first rung 222, such that the angled struts 215 along the first rung 222 define all of the inflow border peak junctions 238 and all of the inflow border through junctions 239, resulting in a zig-zagged pattern of the inflow border 236. Moreover, as shown, the inner frame 210 does not include any other junctions 227 between the inflow border peak junctions 238 and all of the inflow border through junctions 239.

The outflow border 232, in contrast, is defined by struts 214 of more than one row of cells 230, and more specifically, by angled struts 215 of more than one rung, such as strut 215-1 of the uppermost row of cells 230c and the fourth rung 225, strut 215-2 of the second row of cells 230b and the third rung 224, and strut 215-3 of the first or lowermost row of cells 230a or the second rung 223. This results in a generally undulating path formed by the outflow border 232, resulting from the fact that the outflow border peak junctions 234 are formed by struts 214 of the uppermost row of cells 230c or the angled struts 2165 of the uppermost rung, which is the fourth rung 225 in the illustrated example, while the outflow border though junctions 235 are formed by struts 214 of another row of cells 230b or the angled struts 215 of another rung, distal to the uppermost rung, such as the second rung 223 in the illustrated example. This also means that in such examples, the outflow border 232 includes at least one other junction 227 disposed between each outflow border peak junction 234 and a successive outflow border though junction 235, such as junctions 227-1 and 227-2 in the example illustrated in FIG. 7B.

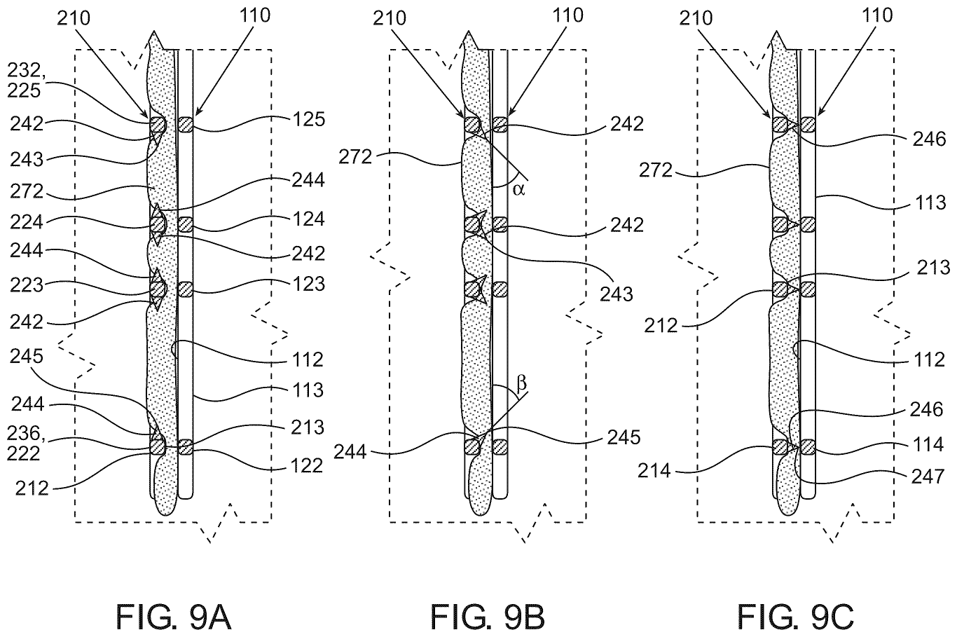

At least some, and preferably all, angled struts 215 of inner frame 210 are provided with spikes 240 extending therefrom. The spikes 240 can include, in examples, distally extending spikes 242 terminating with sharp distal tips 243 (shown also in FIGS. 9A-9B), proximally extending spikes 244 terminating with sharp distal tips 245 (shown also in FIGS. 9A-9B), and in some cases, radially extending spikes 246 terminating with sharp distal tips 247 (shown, for example, in FIG. 9C).

In some examples, the struts 214 forming the outflow border 232 can include distally extending spikes 242 or radially extending spikes 246, but not proximally extending spikes (244). In some examples, the struts 214 forming the inflow border 236 can include proximally extending spikes 244 or radially extending spikes 246, but not distally extending spikes (242). In some examples, struts 214 defined between the outflow border 232 and the inflow border 236 can include proximally extending spikes 244, distally extending spikes 242, radially extending spikes 246, and any combination thereof. For example, strut 215-8 illustrated in FIG. 7B is shown to include both proximally extending spikes 244 and distally extending spikes 242 projecting at opposite directions of the strut.

FIG. 8 shows an example of a prosthetic valve 200 that includes main frame 110, inner frame 210 mounted within main frame 110 and coupled thereto, and valvular structure 260 disposed between main frame 110 and inner frame 210, with spikes 240 of the inner frame 210 engaged with (for example, extending into) valvular structure 260. Valvular structure 260 of FIG. 8 can be implemented, in examples, as valvular structure 260^{a} or valvular structure 260′, such that adjacent leaflets 262 as shown in FIG. 5, or adjacent movable regions 271^{a} as shown in FIG. 4, can be arranged together to form commissures 269 that are coupled (directly or indirectly) to respective portions of the main frame 110, according to any manner described above with respect to prosthetic valve 100 or other types of prosthetic valves. For example, leaflets 262 or movable regions 271^{a} can be secured at their tabs 268^{b} or 268^{a}, to form commissures 269^{b} or 269^{a} of the valvular structure 260^{b} or 260^{a}, respectively, which can be secured, directly or indirectly, to structural elements of main frame 110, such as commissure windows 119.

In examples, at least some of the struts 214 of inner frame 210 are aligned with corresponding struts 114 of main frame 110. For example, inner frame 210 in the illustrated example is configured such that the angled struts 215 of the first rung 222, the second rung 223, the third rung 224, and the fourth rung 225, align with the angled struts 115 of the first rung 122, the second rung 123, the third rung 124, and the fourth rung 125, respectively. Similarly, the axial struts 216 of inner frame 210 align with the distal axial struts 117 of main frame 110.

Similarly, at least some, and optionally all of the cells 230 of the inner frame, are aligned with corresponding cells 130 of the main frame 110. For example, inner frame 210 in the illustrated example is configured such that cells 230a of the first row, cells 230b of the second row, and cells 230c of the third row, align with cells 130a of the first row, cells 130b of the second row, and cells 130c of the third row of the main frame 110. Since all of the rows of cells 130 of the main frame 110 are continuous, while some of the rows of cells 230 of the inner frame 210 are discontinuous, all of the cells of the discontinuous rows of cells of the inner frame, such as along rows of cells 230b and 230c, align with only some of the cells of the continuous rows of cells, namely the second row of cells 130b and the third row of cells 130c, respectively, of the main frame 110.

In this manner, junctions 227 of inner frame 210 can be coupled to junctions 127 of main frame 110, as shown in FIG. 8. Any of the junctions 227 of inner frame 210 can be coupled to corresponding junction 127 of the main frame 110, including any of the inflow border peak junctions 238, inflow border through junctions 239, outflow border peak junction 234, outflow border through junctions 235, and any other junctions 227 disposed between the inflow border 236 and outflow border 232. Similarly, junctions of the main frame 110 to which the inner frame 210 is attached, can refer to any of the inflow apices 129, and any other junctions 127 disposed between the inflow end 102 and the outflow end 101.

While the inner frame is shown in the illustrated examples to generally resemble the shape and/or structure of the main frame, it is to be understood that in other examples, the inner frame can have other shapes and/or structures differing from that of the main frame, such that only a small portion, or none of, the struts and/or cells of both frames are aligned with each other, as long as the inner frame is designed to move between the crimped and expanded states along with the main frame it is coupled to. Moreover, it is to be understood that none of the inner frames and/or main frames illustrated throughout the drawings are limited to these specific shapes of cells and/or struts, and that the same principles can be applied to main frames and inner frame have different shapes and number of cells, rows of cells, struts, and/or rungs of struts.

In the example illustrated in FIG. 8A, the inner frame 210 is coupled to the main frame 110 with sutures 204. In some examples, the inner frame 210 comprises engagements frame junction apertures 231 (see FIG. 7B), that can be formed as openings formed in any of the inner frame's junctions 227, configured to receive the sutures 204. In other examples, the inner frame 210 can be coupled to the main frame 110 in various other ways (for example, fasteners, welding, adhesive, etc.). By coupling the inner frame's junctions 227 to the main frame's junctions 127, the inner frame 210 can, for example, expand and/or contract simultaneously with the main frame 110.

In some examples, the inner frame 210 is removably coupled to the main frame 110 (for example, with sutures 204 and/or fasteners). The term “removably coupled”, as used herein, means coupled in such a way that two components are coupled together and can be separated without plastically deforming either of the components. In some examples, the inner frame 210 can be permanently coupled in such a way that the two components cannot be separated without plastically deforming at least one of the components.

It is to be understood that any reference throughout the current disclosure, to an inner frame being coupled to a main frame, refers to the inner frame being either directly or indirectly coupled to the main frame. For example, an inner frame can be directly coupled to a main frame via sutures (for example, sutures 204) or other coupling means. Alternatively, the inner frame can be indirectly coupled to a main frame, without inclusion of sutures looping around corresponding junctions of the main frame and the inner frame, but rather via an intermediate components, such as the valvular structure. Specifically, the inner frame can be engaged with the valvular structure—such as by spikes of the inner frame effectively attaching the inner frame to the valvular structure, while the valvular structure can be, in turn, attached to the main frame—such as by sutures or any other coupling means, resulting in the inner frame being indirectly coupled to the outer frame via the valvular structure.

The inner frame can be made of any of various suitable plastically-deformable materials (for example, stainless steel, etc.) and/or self-expanding materials (for example, Nitinol). When the inner frame comprises plastically-expandable material, the inner frame (and thus, the prosthetic valve as a whole) can be crimped to a radially compressed state in a delivery shaft and then expanded inside a patient by an inflatable balloon or equivalent expansion mechanism of the delivery apparatus. When the inner frame comprises self-expandable materials, the inner frame (and thus the prosthetic valve as a whole) can be crimped to a radially compressed state and restricted in the compressed state by a shaft, a capsule or equivalent mechanism of the delivery apparatus. Once inside the body, the prosthetic valve can be advanced from the delivery shaft, which allows the prosthetic valve to expand to a partially expanded diameter, and further to the final functional diameter. Suitable plastically-deformable materials can include, but are not limited to, stainless steel, a nickel based alloy (for example, a cobalt-chromium or a nickel-cobalt-chromium alloy such as MP35N alloy), polymers, or combinations thereof.

In some examples, the outflow border 232 of the inner frame 210 is aligned with the proximal attachment line 264 of the valvular structure 260. The inner frame 210 is designed to align with the engagement portion 272, such that the engagement portion 272 is the portion of the valvular structure 260 disposed between the inner frame 210 and the main frame 110, while the movable portion 270 is defined as the portion of the valvular structure 260 proximal to the outflow border 232 (for example, between outflow border 232 and outflow end 201), which is not aligned with struts of the inner frame.

FIGS. 9A-9C shows different arrangements of spikes 240 extending from struts 214 of the inner frame 210, illustrated in sectional views along line 9-9 taken from FIG. 8. FIGS. 9A and 9B shows variations of distally extending spikes 242 and proximally extending spikes 244, extending from struts 214 of the inner frame 210, wherein (1) angled struts 215 of the outflow border 232, such as the struts of the fourth rung 225, include distally extending spikes 242 and are devoid of proximally extending spikes (244); (2) angled struts 215 of the inflow border 236, such as the struts of the first rung 222, include proximally extending spikes 244 and are devoid of distally extending spikes (242); and (3) angled struts 215 disposed between the outflow border 232 and the inflow border 236, such as struts of the third rung 224 and the second rung 223, include both distally extending spikes 242 and proximally extending spikes 244.

In examples, at least some of the spikes 240, and optionally all of the spikes 240, extend solely in the axial direction (i.e., parallel to central axis L) and not in a radial direction. For example, both distally extending spikes 242 and proximally extending spikes 244 extend solely in the axial direction, disposed entirely between the inner surface 212 and outer surface 213 of the inner frame 210. The valvular structure 260, and more specifically, the engagement portion 272, can comprise a compressible material, such as natural tissue (for example, pericardial tissue) or a compressible synthetic material, which can be pressed between the main frame 110 and the inner frame 210 so as to bulge radially inward between the struts 214 of the inner frame 210. In this manner, as illustrated, sharp distal tips of the spikes 240, such as sharp distal tips 243 of distally extending spikes 242 and sharp distal tips 245 of proximally extending spiked 244 can penetrate into the bulging engagement portion.

In examples, at least some of the spikes 240, and optionally all of the spikes 240 are angled spikes that extend both in the axial direction axially and radially outward. For example, as shown in FIG. 9B, the distally extending spikes 242 can extend at an angle axially in the distal direction, and radially outward, toward main frame 110. Similarly, proximally extending spikes 244 can extend at an angle axially in the proximal direction, and radially outward, toward main frame 110.

Stated otherwise, a sharp angle α (shown for example in FIG. 9B) can be defined between the annular outer surface 213 defined by the angled struts 215 of the inner frame 210, and each distally extending angled spike 242. The angle α can be similarly defined between each distally extending angled spike 242 and the central longitudinal axis L, meaning that the distally extending angled spikes 242 are not parallel to the central longitudinal axis L or any axis that extends in parallel to the central longitudinal axis L, in such examples. In some examples, the angle α is in the range of 10-80 degrees. In some examples, the angle α is in the range of 20-70 degrees. In some examples, the angle α is in the range of 30-60 degrees.

Similarly, a sharp angle β (also shown for example in FIG. 9B) can be defined between the annular outer surface 213 defined by the angled struts 215 of the inner frame 210, and each proximally extending angled spike 244. The angle β can be similarly defined between each proximally extending angled spike 244 and the central longitudinal axis L, meaning that the proximally extending angled spikes 244 are not parallel to the central longitudinal axis L or any axis that extends in parallel to the central longitudinal axis, in such examples. The angle β can be similar to, or different from, the angle α. In some examples, the angle β is in the range of 10-80 degrees. In some examples, the angle β is in the range of 20-70 degrees. In some examples, the angle β is in the range of 30-60 degrees.

In examples, at least some of the spikes 240, and optionally all of the spikes 240, extend radially outward without extending in an axial direction. FIG. 9C shows an example in which all of the struts 214 of the inner frame 210, including angled struts 215 of the outflow border 232, such as the struts of the fourth rung 225, angled struts 215 of the inflow border 236, such as the struts of the first rung 222, and angled struts 215 disposed between the outflow border 232 and the inflow border 236, such as struts of the third rung 224 and the second rung 223, include only radially extending spikes 246, extending toward main frame 110, such that their sharp distal tips 247 can engage with (for example, penetrate into) the regions of the engagement portion 272 aligned with the struts 214 of the inner frame 210.

The spikes 240 of any of the above mentioned configurations are configured to engage (and in some instances penetrate) the engagement portion 272 of the valvular structure 260, and retain engagement with the valvular structure 260 during prosthetic valve deployment and after implantation in the patient's body. As mentioned, in some examples, each spike 240 terminates with a sharp distal tip.

While only angled struts are shown in the illustrated examples to include spikes extending therefrom, it is to be understood that in some examples, axial struts can also include spikes. For example, some or all of the axial struts 216 can include radially extending spikes 246 (example no shown). Furthermore, while all of the struts in the examples of FIG. 9A-9C are shown to include spikes that are generally oriented in the same orientation, such as only axial orientations in FIG. 9A, only angled orientation in FIG. 9B, or only radial orientation in FIG. 9C, it is to be understood that different struts of the inner frame, and in some cases, the same struts of the inner frame, can include any variation or combination of axial spikes, angled spikes, and/or radially extending spikes.

The movable portion 270 of valvular structure 260 articulates along the outflow border 232 between the closed and open states of the valvular structure 260 during operation of the prosthetic valve within the patient's body (after implantation). Advantageously, when the struts of the outflow border 232 are devoid, in selected examples, of proximally extending spikes (244), risk of tissue (or any other material the valvular structure comprises) tearing is minimized. In contrast, examples in which angled struts 215 which are distal to the outflow border 232, include both distally extending spikes 242 and proximally extending spikes 244 (implemented either as solely axially extending spikes, as illustrated in FIG. 9A, and/or as angles spikes, as illustrated in FIG. 9B) can be advantageous in that the oppositely oriented spikes of such struts can improve engagement with the engagement portion 272 of the valvular structure 260.

In some examples, the prosthetic valve 200 can be devoid of a separate inner skirt, as the valvular structure 260 can be disposed along the inner surface 112 of main frame 110 in a manner that will perform as an inner-skirt replacement.

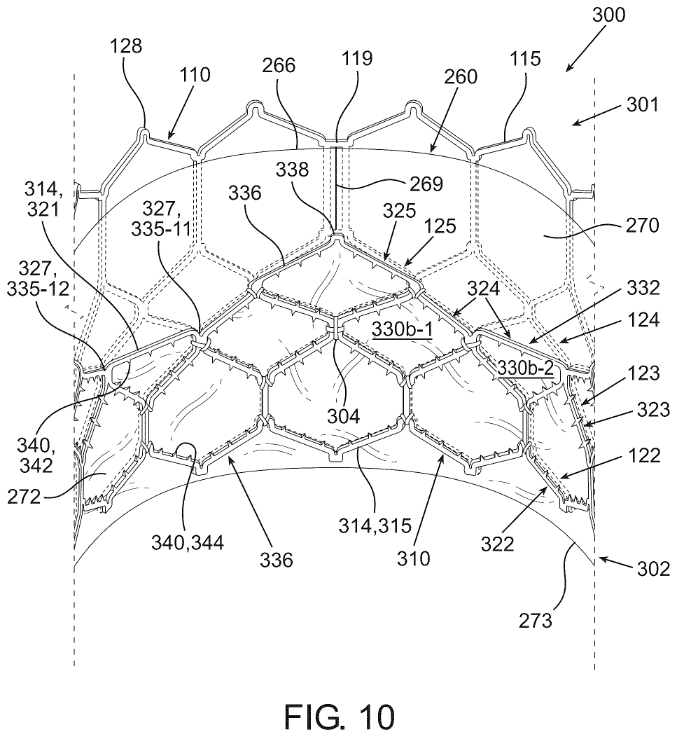

In some examples, the inner frame can include laterally extendable struts. In some examples, the outflow border of an inner frame includes both angles struts and laterally extendable struts. FIG. 10 shows another examples of a prosthetic valve 300 which is similar to any example of prosthetic valve 200 with like reference numerals referring to like components thereof, except that it includes an inner frame 310 instead of inner frame 210. Inner frame 310 is similar to any example of inner frame 210 described above, with like reference numerals referring to like components thereof, except that inner frame 310 can further include laterally extendable struts 321.

A laterally extendable strut 321 is a strut 314 that can assume a generally horizontal or near-horizontal configuration, extending along most of its length (for example, at least 80% of its length) along the circumference of the inner frame 310 in its expanded state such that both ends thereof, terminating at end junctions 327, are axially aligned at the same level between the inflow end and the outflow end. FIG. 10 shows the outflow border 332 comprising a plurality of angled struts 315 and a plurality of laterally extendable struts 321. In particular, at least one rung of struts 314 of the inner frame 310 includes both angled struts 315 and laterally extendable struts 321.

In the illustrated example, a third rung 324, which is a rung distal to the uppermost rung 325, includes angled struts 315 and laterally extendable struts 321. This also results in a corresponding row of cells of the inner frame, namely the second row of cells 330b, including at least two types of cells: diamond or rhombus-shaped cells 330b-1, and triangularly-shaped cells 330b-2. This configuration results in a substantially linear portion of the outflow border 332 formed, by the laterally extendable strut 321, between successive outflow border through junctions 335, instead of a general zig-zagged pattern in the case of inner frame 210 illustrated in FIG. 7B, for example. Thus, the resulting shape of the outflow border 336 is more similar to the generally smoother wave-like pattern of a scalloped line (105) of conventional prosthetic valves (100). While substantially linear laterally extendable strut 321 are illustrated in FIG. 10, it is to be understood that in some examples, the laterally extendable strut 321 can assume an arcuate or bow-like shape (not shown) in an expanded state of the valve, keeping both ends laterally aligned at the same axial level at all times.

In such examples, not all of the cells 330 of inner frame 310 are completely aligned with corresponding cells 130 of the main frame 110. For example, triangularly shaped cells 330b-2 can have two angled struts 315 thereof aligned with angled struts 115 of corresponding cells 130 of main frame 110, while the laterally extendable struts 321 can extend along the internal space defined by corresponding cells 130 of the main frame 110.

It is to be understood that laterally extendable struts 321 assume a substantially final, relatively linearly horizontal or circumferential orientation, in an expanded state of the inner frame 310 (and hence, of the prosthetic valve 300). However, since the inner frame 310 is designed to transition between crimped and expanded configuration, a laterally extendable strut 321 can be a bendable strut 314, which can assume a bent configuration in a crimped state of the inner frame 310, wherein the bending point can be positioned at its center, between both lateral junctions 327 thereof. In some examples, laterally extendable struts 321 can be made of an appropriately bendable material. In some examples, a central region of the laterally extendable strut 321 can include a weakened portion, such as thinner strut portion, or a cutout, to allow the strut 321 to bend over itself at this region. In some examples, the laterally extendable strut 321 can be made of two shorter strut-portions hinged to each other at the center of the resulting strut 321 (i.e., at their end portions).

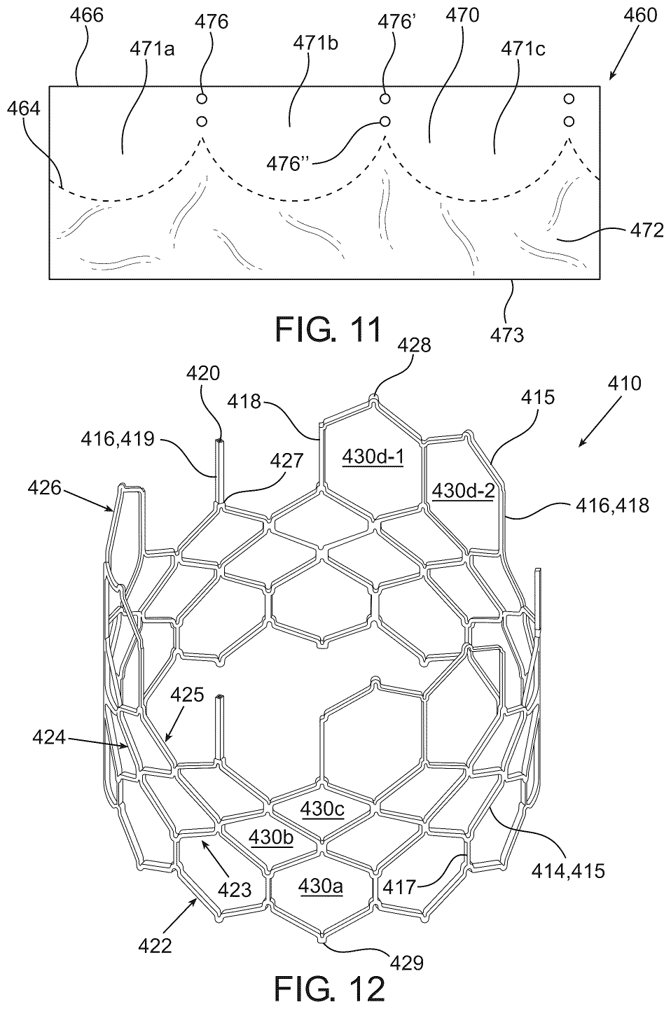

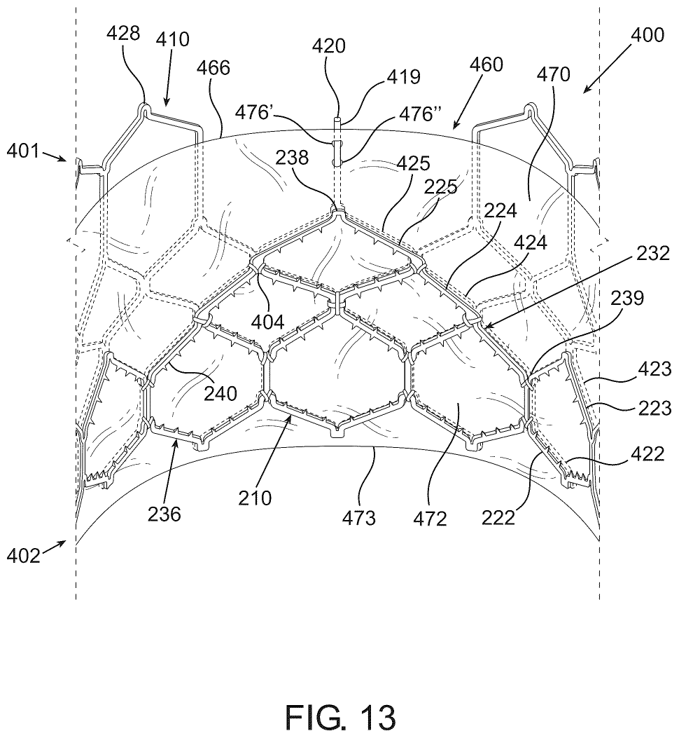

Referring to FIGS. 11-13, a prosthetic valve 400 (shown, for example, in FIG. 13) can include a valvular structure 460 (shown, for example, in FIG. 11) disposed between a main frame 410 (shown, for example, in FIG. 12), and an inner frame which is disposed radially inwardly from, and is coupled to, the main frame 410. While prosthetic valve 400 is illustrated, for example in FIG. 13, in combination with inner frame 210, it is to be understood that it can be similarly utilized with any other type of an inner frame disclosed herein, such as inner frame 310.

The valvular structure 460 can be generally similar to any example described above for valvular structure 260^{a} with respect to FIG. 4, with like reference numerals referring to like components thereof, except that valvular structure 460 can be formed of a unitary piece of material devoid of any tabs. As shown in FIG. 11, the movable portion 470 includes movable regions 471a, 471b and 471c, with at least one aperture 476 disposed between adjacent movable regions 471. In the illustrated example, two apertures 476 are formed between adjacent movable regions 471, such as proximal aperture 476′ positioned closer to free edge 466, and a distal aperture 476″ closer to the proximal attachment line 464.

The main frame 410 can be generally similar to any example described above for main frame 110 with respect to FIG. 1C, with like reference numerals referring to like components thereof, except that instead of commissure windows, some of the axial struts 416, and in particular, some of the proximal axis struts 418, are commissure support struts 419. Commissure support struts 419 are vertically or axially oriented struts which comprise an upper or proximal free end 420. As shown, each commissure support strut 419 is attached at its distal end or junction 427 to two angled struts 415 (for example, angled struts 415 of the fourth rung 425), while its upper free end 420 remains unattached to any other angled strut, meaning that the uppermost or proximal-most row of cells 430d is a discontinuous row of cells, and the uppermost or proximal-most rung of angled struts 415 is a discontinuous rung of struts.

In the illustrated example, each cell 430d is the uppermost row of cells is directly attached to another cell 430d at one side thereof, but is not attached to any other cell 430d of the same row at its opposite side. As shown in FIG. 13, the movable portion 470 of the valvular structure 460 can be mounted over the main frame 410 by extending the commissure support struts 419 through the corresponding apertures 476 of the valvular structure 460. For example, a commissure support struts 419 can extend through two apertures 476′ and 476″ such that a portion of the commissure support struts 419, bound between both apertures 476′, 476″, is disposed over one side of the valvular structure 460, while the remainder of the commissure support struts 419 is disposed over the opposite side of the valvular structure 460. While two apertures 476 are shown between each two adjacent movable regions 471 in the illustrated example, it is to be understood that any other number is contemplated, such as a single aperture, or more than two apertures.

Advantageously, the proposed configuration of a valvular structure 460 allows it to be formed from a unitary piece of material without the need for cutouts along the movable portion 470 for forming tabs, further allowing the valvular structure 460 to follow a relatively linear configuration, and without requiring any of the free edge 466 and/or the engagement portion distal end 473 to follow an arcuate configuration to compensate for spaced-apart tabs.

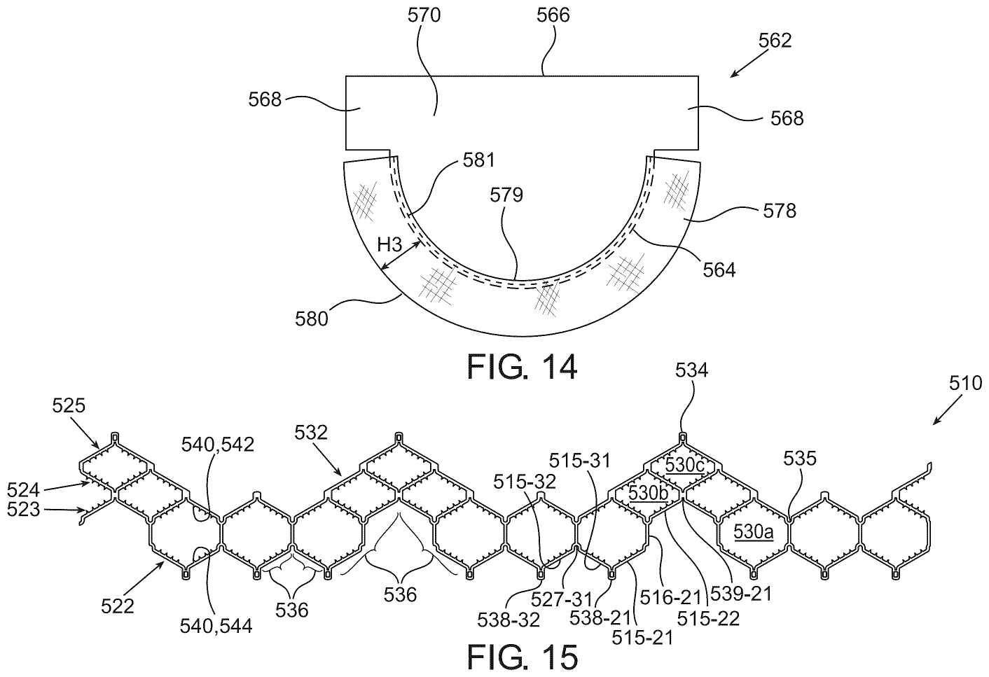

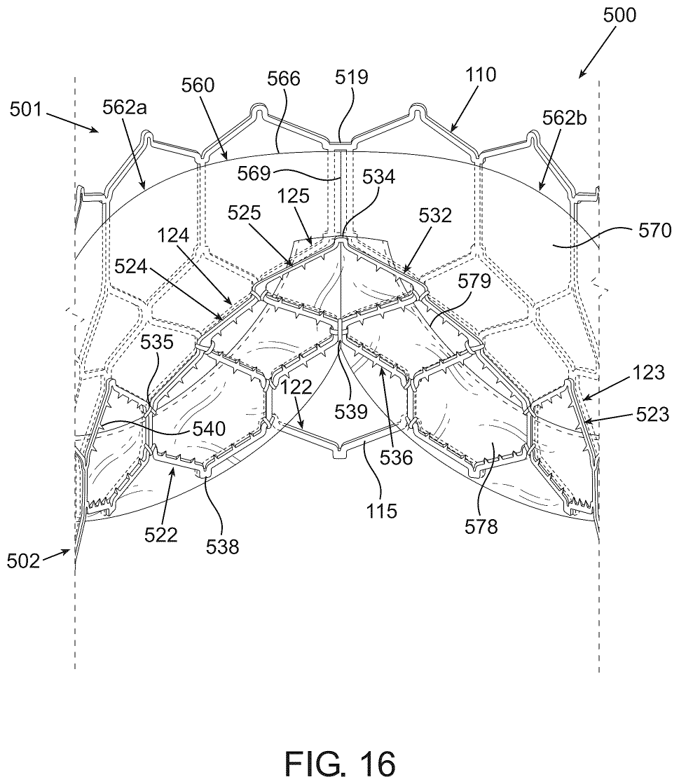

Referring to FIGS. 14-16, a prosthetic valve 500 (shown, for example, in FIG. 16) can include a valvular structure 560 (which can be formed of, for example, leaflets 562 of the type shown in FIG. 14) disposed between a main frame and an inner frame 510 (shown, for example, in FIG. 15) which is disposed radially inwardly from, and is coupled to, the main frame. While prosthetic valve 500 is illustrated, for example in FIG. 16, in combination with main frame 110, it is to be understood that it can be similarly utilized with any other type of a main frame disclosed herein, such as main frame 410.

The valvular structure 560 can be generally similar to any example described above for valvular structures 260 or 460, with like reference numerals referring to like components thereof, except that valvular structure 560 further comprises strip 578 attached to the proximal attachment line, and serving as the engagement portion of valvular structure 560. In some examples, the valvular structure 560 can be formed of a plurality (for example, three) leaflets 562, as shown in FIG. 14. Leaflet 562 can be similar to conventional leaflet 162 described above, including a leaflet body 570 defined between the cusp edge 564 and the free edge 566, with two oppositely directed tabs 568 that can be joined with tabs 568 of adjacent leaflets to form commissure 569. As is the case with leaflet 262 described with respect to FIG. 5, the leaflet body 570 can be also referred to as the movable portion, and the cusp edge 564 can be also referred to as a part of the proximal attachment line. However, unlike leaflet 262, the engagement portion is not necessarily made of the same material as that of the movable portion (either having the same or different material properties, due, for example, to different biological treatment procedures), but is rather attached to a strip 578, which can be also made of natural tissue or a synthetic material, forming the equivalent engagement portion of the leaflet 562.

Strip 578 has a strip proximal edge 579 and a strip distal edge 580, either of which can be, in examples, arcuate, such as by generally tracking or being parallel to the cusp edge 564. The strip 578 is attached to the leaflet body 570 along the strip proximal edge 579 and/or the cusp edge 564, for example by a stitch line 581 sutured parallel to any of the strip proximal edge 579 and/or the cusp edge 564, as shown in FIG. 14. In examples, the strip 578 can be stiffer or more rigid than the leaflet body 570 (i.e., than the movable portion), as described hereinabove with respect to material properties of engagement portion 272 of valvular structure 260, with respect to movable portions 270. In examples, stitch line 581 can define the proximal attachment line of valvular structure 560.

Strip height H3 can be defined between the lowermost point along the cusp edge 564, such as a middle point along cusp edge 564, and a vertically opposing point at the strip distal edge 580. In examples, strip height H3 can be uniform along the length of the strip 578, as shown in FIG. 14. The combination of H1 and H3 results in a total height of the valvular structure 560 (as well as the height of any single leaflet 562), such that the total height of valvular structure 560 and/or leaflet 562 is necessarily greater than an equivalent conventional valvular structure 160 and/or leaflet 162 of the types described above with respect to FIGS. 1A-3. While shown to have a uniform strip height H3, it is to be understood that in alternative examples, strip height H3 can vary along the length of the strip 578, such as from a maximal value at the lowermost portion of the cusp edge 564 to a smaller height H3 closer to tabs 568 (example not shown).

Inner frame 510 is similar to any example of inner frame 210 described above, with like reference numerals referring to like components thereof, except that both the uppermost or proximal-most rung of struts 514 and the lowermost or distal-most rung of struts 514 can be discontinuous rungs of struts. A discontinuous lowermost rungs of struts further means that the lowermost row of cells 530a is also a discontinuous row of cells.

FIG. 15 shows an example of a flattened inner frame 510, which can be identical to inner frame 210 illustrated in FIG. 7B, except that the first rung 522 is a discontinuous rung of angled struts 515, resulting also in the first row of cells 530a being a discontinuous row of cells. In the illustrated example, none of the rows of cells 530 of inner frame 510 is a continuous row of cells. Specifically, while the second row of cells 530b and the third or uppermost row of cells 530c are identical to the equivalent rows of cells 230b and 230c shown in FIG. 7B, the first row of cells 530a can include a total of nine cells 530a, with three of the cells 530a directly connected to adjacent cells 530a of the same row on both of their sides, while the remaining cells 530 are each directly connected to another cell 530a on one side, but is not directly connected to any other cells 530a of the same row on its opposite side.

While the second rung 523, third rung 524, and fourth rung 525 of angled struts 515 of inner frame 510 are similar to the equivalent second rung 223, third rung 224, and fourth rung 225 of inner frame 210 illustrated in FIG. 7B, resulting in an outflow border 532 similarly formed as outflow border 232 of inner frame 210, the inflow border 536 is arranged differently than inflow border 236. In the illustrated example, all angled struts 515 of the first rung 522, selected axial struts 516, and selected angled struts 515 of the second rung 523, define together the inflow border 536 following along a generally undulating or scallop-like path, defining inflow border peak junctions 538 which are the lowermost or distal-most junctions 527 of the inner frame 510, and inflow border through junctions 539 which are the proximal-most junctions 527 along inflow border 536, proximal to inflow border peak junctions 538.

For example, a portion of the inflow border 536 upwardly extending from inflow border peak junction 538-21 to inflow border through junction 539-21 includes an angled strut 515-21 of first rung 522 extending proximally from inflow border peak junction 538-21; an axial strut 516-21 extending therefrom, and an angled strut 515-22 of second rung 523 extending from axial strut 516-21 to inflow border through junction 539-21. A bottom portion of the inflow border 536 is defined by consecutive angled struts 515 of the first rung 522, such as angled strut 515-21, followed by angled strut 515-31 extending from inflow border peak junction 538-21 to junction 527-31, a subsequent angled strut 515-32 extending from junction 527-31 to inflow border peak junction 538-32, and so on, to define three inflow border peak junctions 538 between each consecutive inflow border through junctions 539.

In the illustrated example, inflow border 536 can be defined by struts 514 of more than one row of cells 530, and more specifically, by struts 515 of more than one rung, such as angled strut 515-21 of the first or lowermost row of cells 530a and the first rung 522, axial strut 516-21 of the first or lowermost row of cells 530a, and angled strut 515-22 of the second row of cells 530b or the second rung 523. This results in a generally undulating path formed by the inflow border 536, resulting from the fact that the inflow border peak junctions 538 are formed by struts 514 of the lowermost row of cells 530a or the angled struts 515 of the lowermost rung, which is the first rung 522, while the inflow border though junctions 539 are formed by struts 514 of another row of cells 530b or the angled struts 515 of another rung, distal to the lowermost rung, such as the second rung 523 in the illustrated example. This also means that in such examples, the inflow border 536 includes at least one other junction 527 disposed between each inflow border peak junction 538 and a successive inflow border though junction 539.

As shown in FIG. 16, the undulating shape of the outflow border 532 is designed to generally track the scalloped shape of the strip proximal edge 579 or the stitch line 581, while the undulating shape of inflow border 536 is designed to generally track the scalloped shape of the strip distal edge 580. While shown to include only angled struts 515 and/or axial struts 516, it is to be understood that in examples, inner frame 510 can be adapted to further include laterally extendable struts that can be similar to any example described above for laterally extendable struts 321. For example, the outflow border 532 can be adapted to further include laterally extendable struts, similar to outflow border 332 of inner frame 310 described above, mutatis mutandis. Likewise, while not shown, inflow border 536 can be similarly adapted to further include laterally extendable struts, for example along second rung 523, mutatis mutandis.

While a single leaflet 562 is shown to include a strip 578 having a generally cusp-shaped strip distal edge 580, parallel to cusp edge 564, it other examples, a strip can have a cusp-shaped strip proximal edge 579, as shown in FIG. 14, yet terminate with a relatively linear distal edge, such as engagement portion distal end 273 of leaflet 262^{b} illustrated in FIG. 5. In such examples, the resulting valvular structure (560) can be disposed between a main frame 110 and an inner frame 210, instead of inner frame 510. It is to be understood that the distal edge of a strip 578 can have any other shape besides an arcuate or a linear shape.

While described as being formed of separate leaflets 562, it is to be understood that in alternative examples, the movable portion (570) of valvular structure 560 can be formed of a unitary piece of material, attached (for example, stitched) in a similar manner to a strip (578) defining the engagement portion. For example, valvular structure 560 can include a unitary movable portions (570) similar to any of the movable portion 270^{a} of valvular structure 260^{a} or movable portion 470 of valvular structure 460, with the corresponding engagement portions 272^{a} or 472 replaced by strip 578, wherein the strip (578) can have a distal edge (580) having a generally undulating shape, or alternatively, having an arcuate shape as that of engagement portion distal end 273a or linear configuration as that of engagement portion distal end 473, mutatis mutandis.

As mentioned above, a prosthetic valve can include, in some examples, an outer skirt, such as outer skirt 107 illustrated in FIG. 1A for prosthetic valve 100. In some examples, a valvular structure 660 further includes a skirt 682 attached to its engagement portion distal end 673. The skirt 682 extends from a skirt first end 683, along which it can be attached to the engagement portion 672, and a skirt second end 684, which can be an opposite free end of the skirt 682, The skirt 682 can be made of any material described above with respect to outer skirt 107, and can be sutured to the engagement portion 672 along a stitch line 686, defining a skirt height H4 between the stitch line 686 and the skirt second end 684.

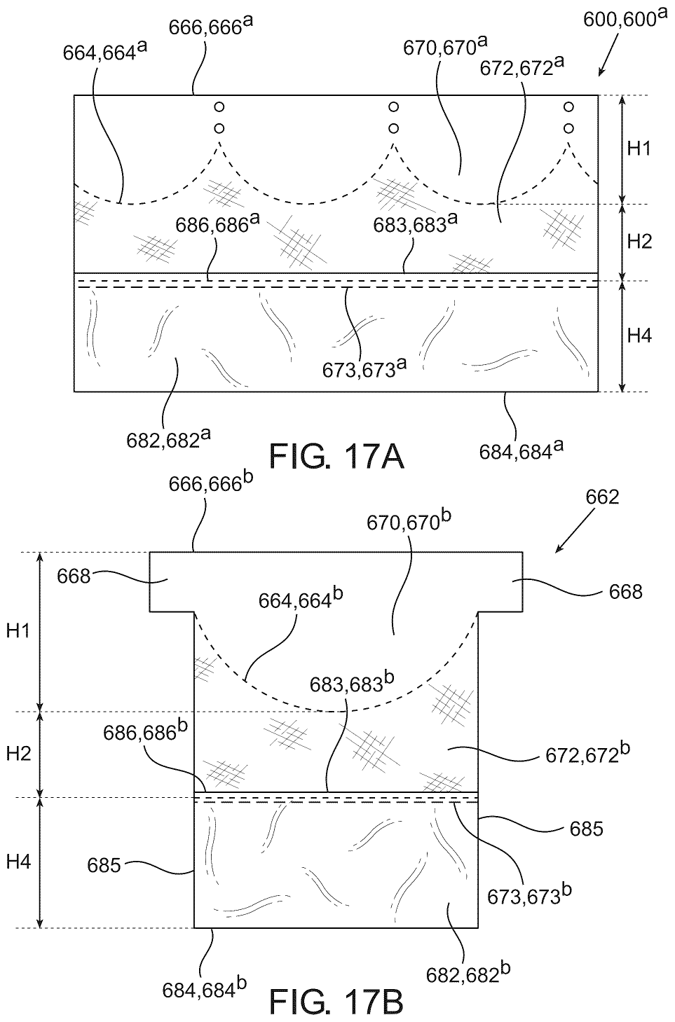

FIG. 17A shows an example of a leaflet structure 660^{a} which can be similar to any example of valvular structure 460 with like reference numerals referring to like components thereof, except that leaflet structure 660a further includes a skirt 682a attached (for example, sutured) to its engagement portion 672^{a}, for example parallel to engagement portion distal end 683^{a}. FIG. 17B shows another example of a single leaflet 662 that can be combined with one or more additional leaflets 662 to form a leaflet structure 660. Leaflet 662 can be similar to any example of leaflet 262 with like reference numerals referring to like components thereof, except that leaflet 662 further includes a skirt 682^{b} attached (for example, sutured) to its engagement portion 672b, for example parallel to engagement portion distal end 683^{b}. When a plurality of leaflet (for example, three) are joined to form a leaflet structure 660^{b}, adjacent skirt portions 682^{b} can be joined (for example, stitched) along their side edges 685 to form together a continuous skirt of the leaflet structure 660^{b}.

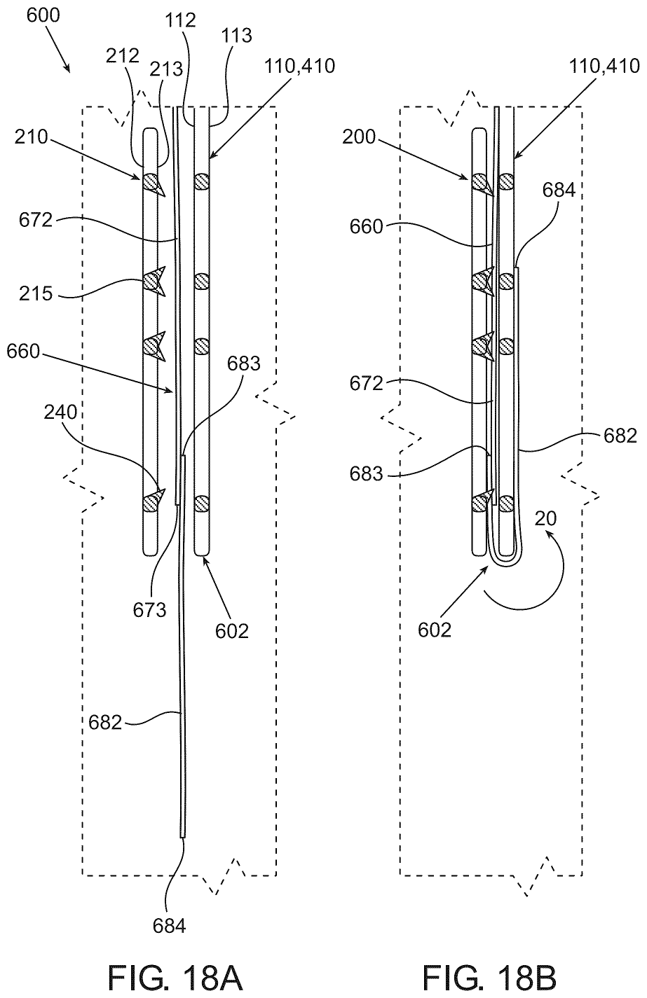

FIGS. 18A-18B shows a prosthetic valve 600, in which, as shown in a partial cross-sectional view in FIG. 18A, the leaflet structure 660 can be placed, during assembly, between the main frame (for example, main frame 110 or 410) and inner frame (for example, inner frame 210) such that the skirt 682 extends beyond inflow end 602. FIG. 18B shows a partial cross-sectional view of a fully assembled prosthetic valve 600, with the leaflet engagement portion 672 sandwiched between the inner and main frames, with spikes 240 engaged therewith (for example, penetrating there-into), wherein the skirt 682 is folded over the inflow end 602 (for example, in direction 20) and is at least partially disposed over the outer surface 113 of main frame 110, and is coupled to main frame 110 (such as by being sutured to struts 114 of main frame 110) to serve as an outer skirt, thereby obviating the need to stitch a separate outer skirt to the main frame 110.

While FIG. 17A illustrates an example of a leaflet structure 660^{a} similar to valvular structure 460, combined with a skirt 682 attached thereto, it is to be understood that a leaflet structure 660 can be similarly based on any other leaflet structure that includes a movable portion formed of a unitary piece of material, such as valvular structure 260^{a} described with respect to FIG. 4, as well as any leaflet structure described above to include a movable portion formed of a unitary piece of material attached to a strip, with the corresponding engagement portion 272^{a} or corresponding strip attached in a similar manner to a skirt 682, mutatis mutandis.

While FIG. 17B illustrates an example of a leaflet 662 similar to leaflet structure 262, combined with a skirt 682 attached thereto, which can be combined with additional leaflets 662 to form valvular structure 660^{b}, it is to be understood that a valvular structure 660 can be similarly based on any other valvular structure that includes separate leaflets, such as leaflet 562 described above to include a strip 578, with the strip 578 attached in a similar manner to a skirt 682 (for example, along strip distal edge 580), mutatis mutandis.

While FIGS. 18A-18B are shown and described above for utilization of a leaflet structure 660 with inner frame 210, it is to be understood that any other type of inner frame can be utilized for forming prosthetic valve 600, including any one of inner frame 310 or inner frame 510, mutatis mutandis. Similarly, any main frame described herein can be used for forming prosthetic valve 600, including any example of main frame 110 or main frame 410.

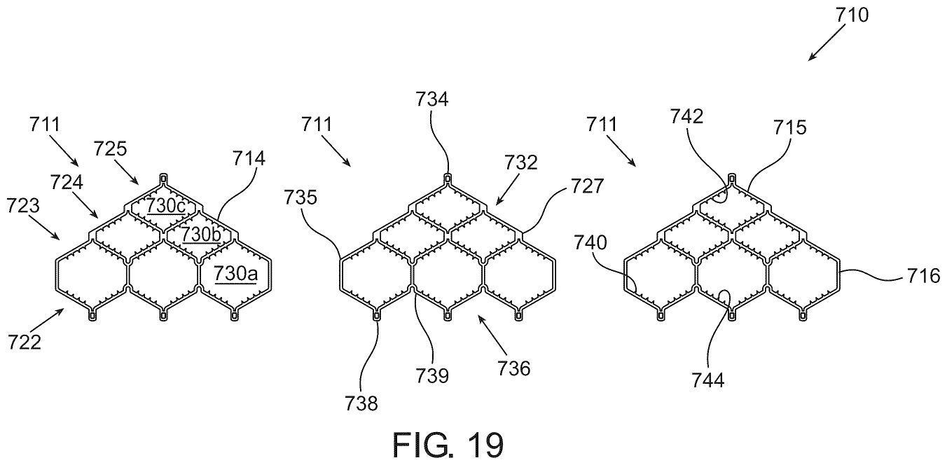

While FIGS. 7B and 15 illustrate examples of inner frames provided as unitary continuous frames, meaning that they assume a unitary circumferential shape substantially similar to that of the main frame, in other examples, the inner frame can be an inner frame assembly formed of a plurality of sub-frame sections. FIG. 19 shows an example of a flattened inner frame assembly 710 that includes three separate sub-frame sections 711, together partially resembling the shape of inner frame 210 illustrated in FIG. 7B, except that the sub-frame sections 711 can be spaced from each other when coupled to the main frame, thereby optionally spanning only part of the perimeter of the main frame.

Specifically, each sub-frame section 711 can include a first rung of struts 722, a second rung of struts 723, a third rung of struts 724 and a fourth rung of struts 725, similar to corresponding rungs 222, 223, 224 and 225 of inner frame 210, except that the total number of struts 714 in some of the rungs of the combined inner frame assembly 710 can be less than the number of struts 214 of corresponding rungs of the unitary continuous inner frame 210. For example, each sub-frame section 711 in the illustrated example includes 6 angled struts 715 in the first rung, resulting in a total of 18 angled struts 715 in the first rung 722 of the combined inner frame assembly 710, which is less than the 24 angled struts 215 of the corresponding first rung 222 of unitary continuous frame 210. Similarly, each sub-frame section 711 in the illustrated example includes 6 angled struts 715 in the second rung, resulting in a total of 18 angled struts 715 in the second rung 723 of the combined inner frame assembly 710, which is less than the 24 angled struts 215 of the corresponding second rung 223 of unitary continuous frame 210.

Each sub-frame section 711 can similarly include a first row of cells 730a, a second row of cells 730b, a third row of cells 730c, similar to corresponding rows of cells 230a, 230b and 230c of inner frame 210, except that the total number of cells 730 in one or more rows of the combined inner frame assembly 710 can be less than the number of cells 230 of corresponding rows of the unitary continuous inner frame 210. For example, each sub-frame section 711 in the illustrated example includes 3 cells in the first row, resulting in a total of 9 cells 730a in the first row of the combined inner frame assembly 710, which is less than the 12 cells 230a of the corresponding first row of unitary continuous frame 210.

It is to be understood that while three sub-frame sections 711 are illustrated, any other number is contemplated, such as two or more than three sub-frame sections that can together define an inner frame assembly. Moreover, while all sub-frame sections 711 are identical to each other in the illustrated example, in alternative examples, one or more sub-frame section can be differently shaped than one other sub-frame section of the inner frame assembly.

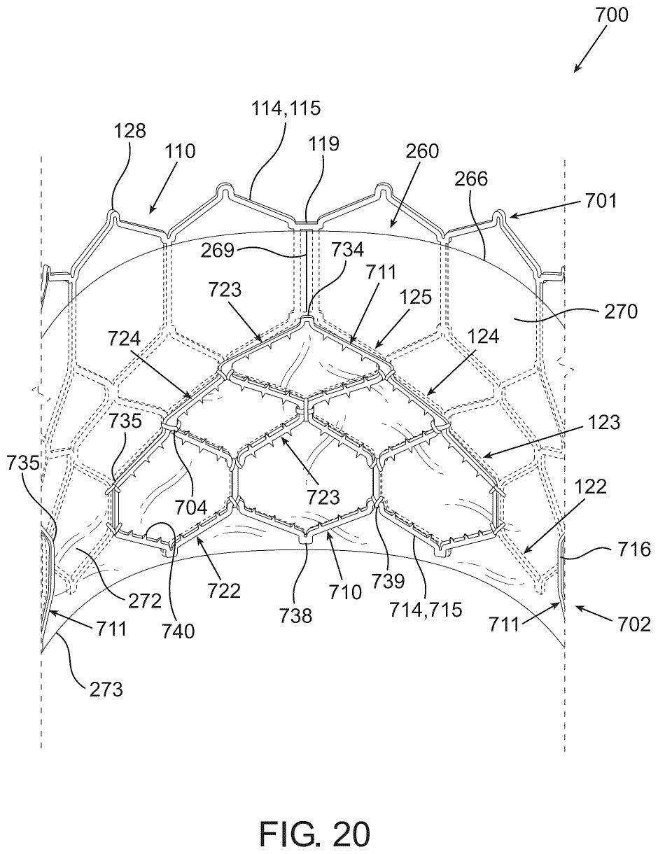

FIG. 20 shows an exemplary prosthetic valve 700 which is similar to any example of prosthetic valve 200 with like reference numerals referring to like components thereof, except that it includes the inner frame assembly 710 instead of inner frame 210. As shown, the sub-frame sections 711 can be laterally spaced from each other when coupled to the main frame 110, wherein the lateral distance between each two adjacent sub-frame sections 711 can be generally equal to the lateral width of a single cell 730a of the first row. This configuration results in all combined rungs of struts 714 of the assembled inner frame 710 being discontinuous rungs, with less struts 714 than the total number of struts 114 of the corresponding rungs of the main frame 110. For example, the first rung 722 of the combined inner frame 710 can include a total of 18 angled struts 715, compared with 24 angled struts 115 of the corresponding first rung 122 of the main frame 110. Similarly, the second rung 723 of the combined inner frame 710 can include a total of 18 angled struts 715, compared with 24 angled struts 115 of the corresponding second rung 123 of the main frame 110. Likewise, all rows of cells 730 of the combined inner frame 710 can include less cells than those of the corresponding rows of the main frame 110. For example, the first row of the combined inner frame 710 can include a total of 9 cells 730a, compared with 12 cells 130a of the corresponding first row of the main frame 110.

While shown to be equi-spaced from each other, it is to be understood that in some examples, some of the sub-frame sections 711 can be unequally spaced from each other, and that the lateral space between adjacent sub-frame sections does not have to be equal to the width of a cell 730a. Moreover, while inner frame assembly 710 is described above and illustrated as having a plurality of sub-frame sections 711 which are spaced from each other when assembled, it is to be understood that in some examples, an inner frame assembly can include a plurality of sub-frame sections which are not spaced from each other when assembled in a prosthetic valve. For example, an inner frame assembly can include a plurality of sub-frame sections which are separate from each other but together can define a shape which is substantially continuous, similar to that of inner frame 210, wherein each sub-frame section can contact, or at least be in close proximity to, the adjacent sub-frame sections, when assembled in a prosthetic valve.

While inner frame assembly 710 is described and illustrated in FIG. 19 to include features which are somewhat similar those of inner frame 210, and is shown in combination with main frame 210 and valvular structure 260 in FIG. 20, it is to be understood that an inner frame assembly can similarly include sub-frame sections that form other structures in their assembled configuration, such as a configuration that can be similar to that described for inner frame 310 with respect to FIG. 10, or inner frame 510 with respect to FIGS. 15-16. And can be used in combination with any example described herein of a main frame, such as main frame 410 described above with respect to FIGS. 12-13, and in combination with any example of valvular structures described herein, such as valvular structure 460 described with respect to FIGS. 11 and 13, valvular structure 560 described above with respect to FIGS. 14 and 16, and/or valvular structure 600 described above with respect to FIGS. 17A-18B, mutatis mutandis.

### Some Examples of the Disclosed Implementation

Some examples of the above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve, comprising:
a main frame comprising a plurality of struts, wherein the main frame is movable between a radially compressed state and a radially expanded state;
an inner frame which is disposed radially inwardly from, and is coupled to, the main frame, the inner frame comprising a plurality of struts and a plurality of spikes extending from at least some of the plurality of struts; and
a valvular structure disposed between the main frame and the inner frame, the valvular structure comprising a movable portion and an engagement portion,
wherein the movable portion is configured to regulate flow through the prosthetic valve;

Example 2. The prosthetic valve of any example herein, particularly example 1, wherein each spike of the plurality of spikes comprises a sharp distal tip configured to penetrate into the engagement portion of the valvular structure.

Example 3. The prosthetic valve of any example herein, particularly example 1 or 2, wherein the spikes comprise distally extending spikes and proximally extending spikes.

Example 4. The prosthetic valve of any example herein, particularly example 3, wherein at least some of the distally extending spikes are extending at a sharp angle α radially outward and in the distal direction from the respective struts.

Example 5. The prosthetic valve of any example herein, particularly example 4, wherein the angle α is in the range of 10-80 degrees.

Example 6. The prosthetic valve of any example herein, particularly example 4, wherein the angle α is in the range of 20-70 degrees.

Example 7. The prosthetic valve of any example herein, particularly example 4, wherein the angle α is in the range of 30-60 degrees.

Example 8. The prosthetic valve of any example herein, particularly any one of examples 3 to 7, wherein at least some of the proximally extending spikes are extending at a sharp angle R radially outward and in the proximal direction from the respective struts.

Example 9. The prosthetic valve of any example herein, particularly example 8, wherein the angle R is in the range of 10-80 degrees.

Example 10. The prosthetic valve of any example herein, particularly example 8, wherein the angle R is in the range of 20-70 degrees.

Example 11. The prosthetic valve of any example herein, particularly example 8, wherein the angle R is in the range of 30-60 degrees.

Example 12. The prosthetic valve of any example herein, particularly any one of examples 1 to 11, wherein the spikes comprise radially extending spikes.

Example 13. The prosthetic valve of any example herein, particularly any one of examples 1 to 12, wherein the struts of the inner frame are arranged in a plurality rungs of struts that comprise a proximal-most rung of struts and a distal-most rung of struts.

Example 14. The prosthetic valve of any example herein, particularly example 13, wherein, when depending on example 3, the proximal-most rung of struts of the inner frame comprises distally extending spikes, and is devoid of proximally extending spikes.

Example 15. The prosthetic valve of any example herein, particularly example 13 or 14, wherein, when depending on example 3, the distal-most rung of struts of the inner frame comprises proximally extending spikes, and is devoid of distally extending spikes.

Example 16. The prosthetic valve of any example herein, particularly any one of examples 13 to 15, wherein the inner frame further comprises at least one additional rung of struts disposed between the proximal-most rung of struts and the distal-most rung of struts.

Example 17. The prosthetic valve of any example herein, particularly example 16, wherein the at least one additional rung of struts comprises at least two additional rungs of struts disposed between the proximal-most rung of struts and the distal-most rung of struts.

Example 18. The prosthetic valve of any example herein, particularly example 16 or 17, wherein the proximal-most rung of struts is a discontinuous rung of struts.

Example 19. The prosthetic valve of any example herein, particularly example 18, wherein, when depending on example 17, at least one of the additional rungs of struts is a discontinuous rung of struts.

Example 20. The prosthetic valve of any example herein, particularly any one of examples 13 to 19, wherein the distal-most rung of struts is a continuous rung of struts.

Example 21. The prosthetic valve of any example herein, particularly any one of examples 1 to 20, wherein the struts of the inner frame define an outflow border comprising outflow border peak junctions and outflow border through junctions, and an inflow border comprising inflow border peak junctions and inflow border through junctions.

Example 22. The prosthetic valve of any example herein, particularly example 21, wherein, when depending on example 16, the outflow border comprises struts of more than one rung of struts.

Example 23. The prosthetic valve of any example herein, particularly example 21 or 22, wherein the outflow border comprises at least one junction disposed between each outflow border peak junction and a successive outflow border through junction.

Example 24. The prosthetic valve of any example herein, particularly any one of examples 1 to 23, wherein the valvular structure comprises a proximal attachment line between the movable portion and the engagement portion.

Example 25. The prosthetic valve of any example herein, particularly example 24, wherein the proximal attachment line comprises a visual marking.

Example 26. The prosthetic valve of any example herein, particularly any one of examples 1 to 25, wherein the engagement portion is stiffer than the movable portion.

Example 27. The prosthetic valve of any example herein, particularly any one of examples 1 to 26, wherein the valvular structure is formed of a unitary piece of material designed to cover the entire circumference of the prosthetic valve, between the main frame and the inner frame.

Example 28. The prosthetic valve of any example herein, particularly example 27, wherein the movable portion comprises a plurality of movable regions.

Example 29. The prosthetic valve of any example herein, particularly example 28, wherein the plurality of movable regions comprises three movable regions.

Example 30. The prosthetic valve of any example herein, particularly example 28 or 29, wherein the valvular structure further comprises two oppositely-directed tabs extending from each movable region, wherein adjacent tabs of each two of the movable regions are joined together to form a commissure, and wherein the commissures are coupled to the main frame.

Example 31. The prosthetic valve of any example herein, particularly example 30, wherein the main frame comprises a plurality of commissure windows, and wherein the commissures are coupled to the corresponding commissure windows.

Example 32. The prosthetic valve of any example herein, particularly example 28 or 29, wherein the valvular structure further comprises at least one aperture disposed between each two adjacent movable regions.

Example 33. The prosthetic valve of any example herein, particularly example 32, wherein the at least one aperture comprises at least two apertures disposed between each two adjacent movable regions.

Example 34. The prosthetic valve of any example herein, particularly example 32 or 33, wherein the main frame comprises a plurality of commissure support struts, each commissure support strut comprising a free end, and wherein the commissure support struts extend through the corresponding apertures of the valvular structure.

Example 35. The prosthetic valve of any example herein, particularly any one of examples 1 to 26, the valvular structure comprises a plurality of leaflets.

Example 36. The prosthetic valve of any example herein, particularly example 35, wherein the plurality of leaflets comprises three leaflets.

Example 37. The prosthetic valve of any example herein, particularly example 35 or 36, wherein each leaflet comprises two oppositely-directed tabs, wherein tabs of adjacent leaflets are joined together to form a commissure, and wherein the commissures are coupled to the main frame.

Example 38. The prosthetic valve of any example herein, particularly example 37, wherein the main frame comprises a plurality of commissure windows, and wherein the commissures are coupled to the corresponding commissure windows.

Example 39. The prosthetic valve of any example herein, particularly any one of examples 35 to 38, wherein the engagement portion of each leaflet comprises a strip attached to the movable portion of the leaflet.

Example 40. The prosthetic valve of any example herein, particularly example 39, wherein the engagement portion of each leaflet comprise a strip attached to the movable portion of the leaflet.

Example 41. The prosthetic valve of any example herein, particularly example 39 or 40, wherein the strip has a uniform strip height.

Example 42. The prosthetic valve of any example herein, particularly example 39 or 21, wherein, when depending on example 16, the inflow border comprises struts of more than one rung of struts.

Example 43. The prosthetic valve of any example herein, particularly any one of examples 21 to 42, wherein the inflow border comprises at least one junction disposed between each inflow border peak junction and a successive inflow border through junction.

Example 44. The prosthetic valve of any example herein, particularly example 16 or 17, wherein the distal-most rung of struts is a discontinuous rung of struts.

Example 45. The prosthetic valve of any example herein, particularly any one of examples 1 to 44, wherein the struts of the inner frame comprise angled struts and laterally extendable struts.

Example 46. The prosthetic valve of any example herein, particularly any one of examples 1 to 45, wherein at least some of the struts of the inner frame are aligned with corresponding struts of the main frame.

Example 47. The prosthetic valve of any example herein, particularly example 46, wherein all of the struts of the inner frame are aligned with corresponding struts of the main frame.

Example 48. The prosthetic valve of any example herein, particularly any one of examples 1 to 47, further comprising a skirt attached to a distal end of the engagement portion.

Example 49. The prosthetic valve of any example herein, particularly example 48, wherein the skirt is folded over an inflow end of the prosthetic valve, and is at least partially disposed over an outer surface of the main frame.

Example 50. The prosthetic valve of any example herein, particularly example 49, wherein the skirt is coupled to the main frame.

Example 51. A prosthetic valve, comprising:
a main frame comprising a plurality of struts defining a plurality of rungs of struts, wherein the main frame is movable between a radially compressed state and a radially expanded state;
an inner frame which is disposed radially inwardly from, and is coupled to, the main frame, the inner frame comprising a plurality of struts defining a plurality of rungs of struts, and a plurality of spikes extending from at least some of the plurality of struts; and
a valvular structure disposed between the main frame and the inner frame;
wherein the plurality of spikes are engaged with the valvular structure.

Example 52. The prosthetic valve of any example herein, particularly example 51, wherein each spike of the plurality of spikes comprises a sharp distal tip configured to penetrate into the valvular structure.

Example 53. The prosthetic valve of any example herein, particularly example 51 or 52, wherein the plurality of rungs of struts of the inner frame comprise: a first rung, a second rung, a third rung, and a fourth rung.

Example 54. The prosthetic valve of any example herein, particularly example 53, wherein the fourth rung of the inner frame is a discontinuous rung of struts.

Example 55. The prosthetic valve of any example herein, particularly example 54, wherein the third rung of the inner frame is a discontinuous rung of struts.

Example 56. The prosthetic valve of any example herein, particularly example 54 or 55, wherein the second rung of the inner frame is a continuous rung of struts.

Example 57. The prosthetic valve of any example herein, particularly example 56, wherein the fourth rung of the inner frame comprises fewer struts than the third rung of the inner frame, and wherein the third rung of the inner frame comprises fewer struts than the second rung of the inner frame.

Example 58. The prosthetic valve of any example herein, particularly example 57, wherein the second rung of the inner frame comprises 24 struts, wherein the third rung of the inner frame comprises 12 struts, and wherein the fourth rung of the inner frame comprises 6 struts.

Example 59. The prosthetic valve of any example herein, particularly any one of examples 53 to 58, wherein the rungs of struts of the main frame comprise: a first rung, a second rung, a third rung, a fourth rung, and a fifth rung.

Example 60. The prosthetic valve of any example herein, particularly example 59, wherein the first rung of the inner frame is aligned with the first rung of the main frame, wherein the second rung of the inner frame is aligned with the second rung of the main frame, wherein the third rung of the inner frame is aligned with the third rung of the main frame, and wherein the fourth rung of the inner frame is aligned with the fourth rung of the main frame.

Example 61. The prosthetic valve of any example herein, particularly example 59 or 60, wherein the struts of the first rung of the main frame intersect at inflow apices.

Example 62. The prosthetic valve of any example herein, particularly any one of examples 59 to 61, wherein all of the rungs of struts of the main frame are continuous rungs of struts.

Example 63. The prosthetic valve of any example herein, particularly example 62, wherein each of the rungs of struts of the main frame comprises 24 struts.

Example 64. The prosthetic valve of any example herein, particularly any one of examples 53 to 62, wherein the struts of the inner frame define an outflow border comprising outflow border peak junctions and outflow border through junctions, and an inflow border comprising inflow border peak junctions and inflow border through junctions.

Example 65. The prosthetic valve of any example herein, particularly example 64, wherein the outflow border peak junctions are defined by intersecting struts of the fourth rung of the inner frame.

Example 66. The prosthetic valve of any example herein, particularly example 65, wherein the outflow border comprises struts of the fourth rung of the inner frame, the third rung of the inner frame, and the second rung of the inner frame.

Example 67. The prosthetic valve of any example herein, particularly example 66, wherein the outflow border through junctions are defined by intersecting struts of the second rung of the inner frame.

Example 68. The prosthetic valve of any example herein, particularly example 66 or 67, wherein the outflow border comprises at least one strut of the fourth rung of the inner frame, at least one strut of the third rung of the inner frame, and at least one strut of the second rung of the inner frame, disposed between each outflow border peak junction and a successive outflow border through junction.

Example 69. The prosthetic valve of any example herein, particularly any one of examples 66 to 68, wherein the outflow border comprises at least two junctions between each outflow border peak junction and a successive outflow border through junction.

Example 70. The prosthetic valve of any example herein, particularly any one of examples 66 to 69, wherein the outflow border comprises at least two outflow border through junctions between each two successive outflow border peak junctions.

Example 71. The prosthetic valve of any example herein, particularly any one of examples 64 to 70, wherein the inflow border peak junctions are defined by intersecting struts of the first rung of the inner frame.

Example 72. The prosthetic valve of any example herein, particularly any one of examples 64 to 71, wherein the first rung of the inner frame is a continuous rung of struts.

Example 73. The prosthetic valve of any example herein, particularly example 72, wherein the first rung of the inner frame comprises 24 struts.

Example 74. The prosthetic valve of any example herein, particularly example 72 or 73, wherein the inflow border through junctions are defined by intersecting struts of the first rung of the inner frame.

Example 75. The prosthetic valve of any example herein, particularly any one of examples 64 to 71, wherein the first rung of the inner frame is a discontinuous rung of struts.

Example 76. The prosthetic valve of any example herein, particularly example 75, wherein the first rung of the inner frame comprises 18 struts.

Example 77. The prosthetic valve of any example herein, particularly example 75 or 76, wherein the inflow border comprises struts of the first rung of the inner frame and the second rung of the inner frame.

Example 78. The prosthetic valve of any example herein, particularly any one of examples 75 to 77, wherein the inflow border through junctions are defined by intersecting struts of the second rung of the inner frame.

Example 79. The prosthetic valve of any example herein, particularly example 74 or 78, wherein the inflow border comprises at least one strut of the first rung of the inner frame, and at least one strut of the second rung of the inner frame, disposed between each inflow border peak junction and a successive outflow border through junction.

Example 80. The prosthetic valve of any example herein, particularly any one of examples 77 to 79, wherein the inflow border comprises at least one junction between each inflow border peak junction and a successive inflow border through junction.

Example 81. The prosthetic valve of any example herein, particularly any one of examples 77 to 80, wherein the inflow border comprises at least three inflow border peak junctions between each two successive inflow border through junctions.

Example 82. The prosthetic valve of any example herein, particularly any one of examples 77 to 81, wherein the inflow border further comprises at least one axial strut between each inflow border peak junction and a successive inflow border through junction.

Example 83. The prosthetic valve of any example herein, particularly any one of examples 64 to 82, wherein the at least some of the struts of the outflow border are laterally extendable struts.

Example 84. The prosthetic valve of any example herein, particularly example 83, wherein the laterally extendable struts are struts of the third rung of the inner frame.

Example 85. The prosthetic valve of any example herein, particularly any one of examples 64 to 83, wherein the spikes comprise distally extending spikes and proximally extending spikes.

Example 86. The prosthetic valve of any example herein, particularly example 85, wherein at least some of the distally extending spikes are extending at a sharp angle α radially outward and in the distal direction from the respective struts.

Example 87. The prosthetic valve of any example herein, particularly example 86, wherein the angle α is in the range of 10-80 degrees.

Example 88. The prosthetic valve of any example herein, particularly example 86, wherein the angle α is in the range of 20-70 degrees.

Example 89. The prosthetic valve of any example herein, particularly example 86, wherein the angle α is in the range of 30-60 degrees.

Example 90. The prosthetic valve of any example herein, particularly any one of examples 85 to 89, wherein at least some of the proximally extending spikes are extending at a sharp angle R radially outward and in the proximal direction from the respective struts.

Example 91. The prosthetic valve of any example herein, particularly example 90, wherein the angle R is in the range of 10-80 degrees.

Example 92. The prosthetic valve of any example herein, particularly example 90, wherein the angle R is in the range of 20-70 degrees.

Example 93. The prosthetic valve of any example herein, particularly example 90, wherein the angle R is in the range of 30-60 degrees.

Example 94. The prosthetic valve of any example herein, particularly any one of examples 85 to 93, wherein the outflow border is devoid of proximally extending spikes.

Example 95. The prosthetic valve of any example herein, particularly example 94, wherein the outflow border comprises distally extending spikes.

Example 96. The prosthetic valve of any example herein, particularly any one of examples 85 to 95, wherein the inflow border is devoid of distally extending spikes.

Example 97. The prosthetic valve of any example herein, particularly example 96, wherein the inflow border comprises proximally extending spikes.

Example 98. The prosthetic valve of any example herein, particularly any one of examples 85 to 97, wherein some of the struts of the inner frame, disposed between the inflow border and the outflow border, comprise both proximally extending spikes and distally extending spikes.

Example 99. The prosthetic valve of any example herein, particularly any one of examples 64 to 98, wherein the spikes comprise radially extending spikes.

Example 100. The prosthetic valve of any example herein, particularly any one of examples 51 to 99, wherein the valvular structure comprises an engagement portion disposed between the inner frame and the main frame, and a movable portion disposed proximal to the outflow border of the inner frame.

Example 102. The prosthetic valve of any example herein, particularly example 100 or 101, wherein the valvular structure is formed of a unitary piece of material designed to cover the entire circumference of the prosthetic valve, between the main frame and the inner frame.

Example 103. The prosthetic valve of any example herein, particularly example 102, wherein the movable portion comprises a plurality of movable regions.

Example 104. The prosthetic valve of any example herein, particularly example 103, wherein the plurality of movable regions comprises three movable regions.

Example 105. The prosthetic valve of any example herein, particularly example 103 or 104, wherein the valvular structure further comprises two oppositely-directed tabs extending from each movable region, wherein adjacent tabs of each two of the movable regions are joined together to form a commissure, and wherein the commissures are coupled to the main frame.

Example 106. The prosthetic valve of any example herein, particularly example 105, wherein the main frame comprises a plurality of commissure windows, and wherein the commissures are coupled to the corresponding commissure windows.

Example 107. The prosthetic valve of any example herein, particularly example 103 or 104, wherein the valvular structure further comprises at least one aperture disposed between each two adjacent movable regions.

Example 108. The prosthetic valve of any example herein, particularly example 107, wherein the at least one aperture comprises two apertures disposed between each two adjacent movable regions.

Example 109. The prosthetic valve of any example herein, particularly example 107 or 108, wherein the main frame comprises a plurality of commissure support struts, each commissure support strut comprising a free end, and wherein the commissure support struts extend through the corresponding apertures of the valvular structure.

Example 110. The prosthetic valve of any example herein, particularly example 100 or 101, wherein the valvular structure comprises a plurality of leaflets.

Example 111. The prosthetic valve of any example herein, particularly example 110, wherein the plurality of leaflets comprises three leaflets.

Example 112. The prosthetic valve of any example herein, particularly example 110 or 111, wherein each leaflet comprises two oppositely-directed tabs, wherein tabs of adjacent leaflets are joined together to form a commissure, and wherein the commissures are coupled to the main frame.

Example 113. The prosthetic valve of any example herein, particularly example 112, wherein the main frame comprises a plurality of commissure windows, and wherein the commissures are coupled to the corresponding commissure windows.

Example 114. The prosthetic valve of any example herein, particularly any one of examples 110 to 113, wherein the engagement portion of each leaflet comprise a strip attached to the movable portion of the leaflet.

Example 115. The prosthetic valve of any example herein, particularly example 114, wherein the strip is sutured to a cusp edge of the movable portion of the leaflet, along a stitch line.

Example 116. The prosthetic valve of any example herein, particularly example 114 or 115, wherein the strip has a uniform strip height.

Example 117. The prosthetic valve of any example herein, particularly any one of examples 100 to 116, further comprising a skirt attached to a distal end of the engagement portion.

Example 118. The prosthetic valve of any example herein, particularly example 114 or 117, wherein the skirt is folded over an inflow end of the prosthetic valve, and is at least partially disposed over an outer surface of the main frame.

Example 119. The prosthetic valve of any example herein, particularly example 114 or 118, wherein the skirt is coupled to the main frame.

Example 120. A prosthetic valve comprising:
a main frame comprising a plurality of struts defining a plurality of rows of cells, wherein the main frame is movable between a radially compressed state and a radially expanded state;
an inner frame which is disposed radially inwardly from, and is coupled to, the main frame, the inner frame comprising a plurality of struts defining a plurality of rows of cells, and a plurality of spikes extending from at least some of the plurality of struts; and
a valvular structure disposed between the main frame and the inner frame;
wherein the plurality of spikes are engaged with the valvular structure.

Example 121. The prosthetic valve of any example herein, particularly example 120, wherein each spike of the plurality of spikes comprises a sharp distal tip configured to penetrate into the valvular structure.

Example 122. The prosthetic valve of any example herein, particularly example 121 or 122, wherein the plurality of rows of cells of the inner frame comprise: a first row, a second row, and a third row.

Example 123. The prosthetic valve of any example herein, particularly example 122, wherein the third row of the inner frame is a discontinuous row of cells.

Example 124. The prosthetic valve of any example herein, particularly example 123, wherein the second row of the inner frame is a discontinuous row of cells.

Example 125. The prosthetic valve of any example herein, particularly example 124, wherein the third row of the inner frame comprises fewer cells than the second row of the inner frame.

Example 126. The prosthetic valve of any example herein, particularly example 125, wherein the third row of the inner frame comprises 3 cells, and wherein the second row of the inner frame comprises 6 cells.

Example 127. The prosthetic valve of any example herein, particularly any one of examples 122 to 126, wherein the rows of cells of the main frame comprise: a first row, a second row, a third row, and a fourth row.

Example 128. The prosthetic valve of any example herein, particularly example 127, wherein the first row of the inner frame is aligned with the first row of the main frame, wherein the second row of the inner frame is aligned with the second row of the main frame, and wherein the third row of the inner frame is aligned with the third row of the main frame.

Example 129. The prosthetic valve of any example herein, particularly example 127 or 128, wherein struts of cells of the first row of the main frame intersect at inflow apices.

Example 130. The prosthetic valve of any example herein, particularly any one of examples 127 to 129, wherein all of the rows of cells of the main frame are continuous rows of cells.

Example 131. The prosthetic valve of any example herein, particularly example 130, wherein each of the rows of cells of the main frame comprises 12 cells.

Example 132. The prosthetic valve of any example herein, particularly any one of examples 122 to 131, wherein the struts of the inner frame define an outflow border comprising outflow border peak junctions and outflow border through junctions, and an inflow border comprising inflow border peak junctions and inflow border through junctions.

Example 133. The prosthetic valve of any example herein, particularly example 132, wherein the outflow border peak junctions are defined by intersecting struts of cells of the third row of the inner frame.

Example 134. The prosthetic valve of any example herein, particularly example 133, wherein the outflow border comprises struts of cells of the third row of the inner frame, the second row of the inner frame, and the first row of the inner frame.

Example 135. The prosthetic valve of any example herein, particularly example 134, wherein the outflow border through junctions are defined by intersecting struts of cells of the first row of the inner frame.

Example 136. The prosthetic valve of any example herein, particularly example 134 or 135, wherein the outflow border comprises at least one strut of a cell the third row of the inner frame, at least one strut of a cell of the second row of the inner frame, and at least one strut of a cell of the first row of the inner frame, disposed between each outflow border peak junction and a successive outflow border through junction.

Example 137. The prosthetic valve of any example herein, particularly any one of examples 134 to 136, wherein the outflow border comprises at least two junctions between each outflow border peak junction and a successive outflow border through junction.

Example 138. The prosthetic valve of any example herein, particularly any one of examples 134 to 137, wherein the outflow border comprises at least two outflow border through junctions between each two successive outflow border peak junctions.

Example 139. The prosthetic valve of any example herein, particularly any one of examples 132 to 138, wherein the inflow border peak junctions are defined by intersecting struts of cells of the first row of the inner frame.

Example 140. The prosthetic valve of any example herein, particularly any one of examples 132 to 139, wherein the first row of the inner frame is a continuous row of cells.

Example 141. The prosthetic valve of any example herein, particularly example 140, wherein the first row of the inner frame comprises 12 cells.

Example 142. The prosthetic valve of any example herein, particularly example 140 or 141, wherein the inflow border through junctions are defined by intersecting struts of cells of the first row of the inner frame.

Example 143. The prosthetic valve of any example herein, particularly any one of examples 132 to 139, wherein the first row of the inner frame is a discontinuous row of cells.

Example 144. The prosthetic valve of any example herein, particularly example 143, wherein the first row of the inner frame comprises 9 cells.

Example 145. The prosthetic valve of any example herein, particularly example 143 or 144, wherein the inflow border comprises struts of cells of the first row of the inner frame and the second row of the inner frame.

Example 146. The prosthetic valve of any example herein, particularly any one of examples 143 to 145, wherein the inflow border through junctions are defined by intersecting struts of cells of the second row of the inner frame.

Example 147. The prosthetic valve of any example herein, particularly example 145 or 146, wherein the inflow border comprises at least one strut of a cell of the first row of the inner frame, and at least one strut of a cell of the second row of the inner frame, disposed between each inflow border peak junction and a successive outflow border through junction.

Example 148. The prosthetic valve of any example herein, particularly any one of examples 145 to 147, wherein the inflow border comprises at least one junction between each inflow border peak junction and a successive inflow border through junction.

Example 149. The prosthetic valve of any example herein, particularly any one of examples 145 to 147, wherein the inflow border comprises at least three inflow border peak junctions between each two successive inflow border through junctions.

Example 150. The prosthetic valve of any example herein, particularly any one of examples 145 to 149, wherein the inflow border further comprises at least one axial strut of a cell of the first row of the inner frame, between each inflow border peak junction and a successive inflow border through junction.

Example 151. The prosthetic valve of any example herein, particularly any one of examples 132 to 150, wherein the at least some of the struts of the outflow border are laterally extendable struts.

Example 152. The prosthetic valve of any example herein, particularly example 151, wherein the laterally extendable struts are struts of a third rung of the inner frame.

Example 153. The prosthetic valve of any example herein, particularly any one of examples 132 to 152, wherein the spikes comprise distally extending spikes and proximally extending spikes.

Example 154. The prosthetic valve of any example herein, particularly example 153, wherein at least some of the distally extending spikes are extending at a sharp angle α radially outward and in the distal direction from the respective struts.

Example 155. The prosthetic valve of any example herein, particularly example 154, wherein the angle α is in the range of 10-80 degrees.

Example 156. The prosthetic valve of any example herein, particularly example 154, wherein the angle α is in the range of 20-70 degrees.

Example 157. The prosthetic valve of any example herein, particularly example 154, wherein the angle α is in the range of 30-60 degrees.

Example 158. The prosthetic valve of any example herein, particularly any one of examples 153 to 157, wherein at least some of the proximally extending spikes are extending at a sharp angle β radially outward and in the proximal direction from the respective struts.

Example 159. The prosthetic valve of any example herein, particularly example 158, wherein the angle β is in the range of 10-80 degrees.

Example 160. The prosthetic valve of any example herein, particularly example 158, wherein the angle β is in the range of 20-70 degrees.

Example 161. The prosthetic valve of any example herein, particularly example 158, wherein the angle β is in the range of 30-60 degrees.

Example 162. The prosthetic valve of any example herein, particularly any one of examples 153 to 161, wherein the outflow border is devoid of proximally extending spikes.

Example 163. The prosthetic valve of any example herein, particularly example 162, wherein the outflow border comprises distally extending spikes.

Example 164. The prosthetic valve of any example herein, particularly any one of examples 153 to 163, wherein the inflow border is devoid of distally extending spikes.

Example 165. The prosthetic valve of any example herein, particularly example 164, wherein the inflow border comprises proximally extending spikes.

Example 166. The prosthetic valve of any example herein, particularly any one of examples 153 to 165, wherein some of the struts of the inner frame, disposed between the inflow border and the outflow border, comprise both proximally extending spikes and distally extending spikes.

Example 167. The prosthetic valve of any example herein, particularly any one of examples 132 to 166, wherein the spikes comprise radially extending spikes.

Example 168. The prosthetic valve of any example herein, particularly any one of examples 132 to 167, wherein the valvular structure comprises an engagement portion disposed between the inner frame and the main frame, and a movable portion disposed proximal to the outflow border of the inner frame.

Example 169. The prosthetic valve of any example herein, particularly example 168, wherein the engagement portion is stiffer than the movable portion.

Example 170. The prosthetic valve of any example herein, particularly example 168 or 169, wherein the valvular structure is formed of a unitary piece of material designed to cover the entire circumference of the prosthetic valve, between the main frame and the inner frame.

Example 171. The prosthetic valve of any example herein, particularly example 170, wherein the movable portion comprises a plurality of movable regions.

Example 172. The prosthetic valve of any example herein, particularly example 171, wherein the plurality of movable regions comprises three movable regions.

Example 173. The prosthetic valve of any example herein, particularly example 171 or 172, wherein the valvular structure further comprises two oppositely-directed tabs extending from each movable region, wherein adjacent tabs of each two of the movable regions are joined together to form a commissure, and wherein the commissures are coupled to the main frame.

Example 174. The prosthetic valve of any example herein, particularly example 173, wherein the main frame comprises a plurality of commissure windows, and wherein the commissures are coupled to the corresponding commissure windows.

Example 175. The prosthetic valve of any example herein, particularly example 171 or 172, wherein the valvular structure further comprises at least one aperture disposed between each two adjacent movable regions.

Example 176. The prosthetic valve of any example herein, particularly example 175, wherein the at least one aperture comprises two apertures disposed between each two adjacent movable regions.

Example 177. The prosthetic valve of any example herein, particularly example 175 or 176, wherein the main frame comprises a plurality of commissure support struts, each commissure support strut comprising a free end, and wherein the commissure support struts extend through the corresponding apertures of the valvular structure.

Example 178. The prosthetic valve of any example herein, particularly example 168 or 169, wherein the valvular structure comprises a plurality of leaflets.

Example 179. The prosthetic valve of any example herein, particularly example 178, wherein the plurality of leaflets comprises three leaflets.

Example 180. The prosthetic valve of any example herein, particularly example 178 or 179, wherein each leaflet comprises two oppositely-directed tabs, wherein tabs of adjacent leaflets are joined together to form a commissure, and wherein the commissures are coupled to the main frame.

Example 181. The prosthetic valve of any example herein, particularly example 180, wherein the main frame comprises a plurality of commissure windows, and wherein the commissures are coupled to the corresponding commissure windows.

Example 182. The prosthetic valve of any example herein, particularly any one of examples 178 to 181, wherein the engagement portion of each leaflet comprise a strip attached to the movable portion of the leaflet.

Example 183. The prosthetic valve of any example herein, particularly example 182, wherein the strip is sutured to a cusp edge of the movable portion of the leaflet, along a stitch line.

Example 184. The prosthetic valve of any example herein, particularly example 182 or 183, wherein the strip has a uniform strip height.

Example 185. The prosthetic valve of any example herein, particularly any one of examples 168 to 184, further comprising a skirt attached to a distal end of the engagement portion.

Example 186. The prosthetic valve of any example herein, particularly example 185, wherein the skirt is folded over an inflow end of the prosthetic valve, and is at least partially disposed over an outer surface of the main frame.

Example 187. A prosthetic valve comprising:
a main frame comprising a plurality of struts, wherein the main frame is movable between a radially compressed state and a radially expanded state;
an inner frame assembly comprising a plurality of sub-frame sections, each sub-frame section disposed radially inwardly from, and is coupled to, the main frame, wherein each sub-frame section comprises a plurality of struts and a plurality of spikes extending from at least some of the plurality of struts; and
a valvular structure disposed between the main frame and the sub-frame sections, the valvular structure comprising a movable portion and an engagement portion, wherein the movable portion is configured to regulate flow through the prosthetic valve;
wherein the plurality of spikes are engaged with the engagement portion of the valvular structure.

Example 188. The prosthetic valve of any example herein, particularly example 187, wherein each spike of the plurality of spikes comprises a sharp distal tip configured to penetrate into the engagement portion of the valvular structure.

Example 189. The prosthetic valve of any example herein, particularly example 187 or 188, wherein the spikes comprise distally extending spikes and proximally extending spikes.

Example 190. The prosthetic valve of any example herein, particularly example 189, wherein at least some of the distally extending spikes are extending at a sharp angle α radially outward and in the distal direction from the respective struts.

Example 191. The prosthetic valve of any example herein, particularly example 190, wherein the angle α is in the range of 10-80 degrees.

Example 192. The prosthetic valve of any example herein, particularly example 190, wherein the angle α is in the range of 20-70 degrees.

Example 193. The prosthetic valve of any example herein, particularly example 190, wherein the angle α is in the range of 30-60 degrees.

Example 194. The prosthetic valve of any example herein, particularly any one of examples 189 to 193, wherein at least some of the proximally extending spikes are extending at a sharp angle R radially outward and in the proximal direction from the respective struts.

Example 195. The prosthetic valve of any example herein, particularly example 194, wherein the angle R is in the range of 10-80 degrees.

Example 196. The prosthetic valve of any example herein, particularly example 194, wherein the angle R is in the range of 20-70 degrees.

Example 197. The prosthetic valve of any example herein, particularly example 194, wherein the angle R is in the range of 30-60 degrees.

Example 198. The prosthetic valve of any example herein, particularly any one of examples 187 to 197, wherein the spikes comprise radially extending spikes.

Example 199. The prosthetic valve of any example herein, particularly any one of examples 187 to 198, wherein the struts of each sub-frame section are arranged in a plurality rungs of struts that comprise a proximal-most rung of struts and a distal-most rung of struts.

Example 200. The prosthetic valve of any example herein, particularly example 199, wherein, when depending on example 189, the proximal-most rung of struts of each sub-frame section comprises distally extending spikes, and is devoid of proximally extending spikes.

Example 201. The prosthetic valve of any example herein, particularly example 199 or 200, wherein, when depending on example 189, the distal-most rung of struts of each sub-frame section comprises proximally extending spikes, and is devoid of distally extending spikes.

Example 202. The prosthetic valve of any example herein, particularly any one of examples 199 to 201, wherein each sub-frame section further comprises at least one additional rung of struts disposed between the proximal-most rung of struts and the distal-most rung of struts.

Example 203. The prosthetic valve of any example herein, particularly example 202, wherein the at least one additional rung of struts comprises at least two additional rungs of struts disposed between the proximal-most rung of struts and the distal-most rung of struts.

Example 204. The prosthetic valve of any example herein, particularly any one of examples 187 to 203, wherein the plurality of sub-frame sections comprises three sub-frame sections.

Example 205. The prosthetic valve of any example herein, particularly any one of examples 187 to 204, wherein the sub-frame sections are laterally spaced from each other.

Example 206. The prosthetic valve of any example herein, particularly any one of examples 187 to 205, wherein the combined number of struts of corresponding rungs of the plurality of sub-frame sections is less than the number of struts of a corresponding rung of the main frame, for all of the rungs of the sub-frame sections.

Example 207. The prosthetic valve of any example herein, particularly any one of examples 187 to 206, wherein the valvular structure comprises a proximal attachment line between the movable portion and the engagement portion.

Example 208. The prosthetic valve of any example herein, particularly example 207, wherein the proximal attachment line comprises a visual marking.

Example 209. The prosthetic valve of any example herein, particularly any one of examples 187 to 208, wherein the engagement portion is stiffer than the movable portion.

Example 210. The prosthetic valve of any example herein, particularly any one of examples 187 to 209, wherein the valvular structure is formed of a unitary piece of material designed to cover the entire circumference of the prosthetic valve, between the main frame and the inner frame.

Example 211. The prosthetic valve of any example herein, particularly example 210, wherein the movable portion comprises a plurality of movable regions.

Example 212. The prosthetic valve of any example herein, particularly example 211, wherein the plurality of movable regions comprises three movable regions.

Example 213. The prosthetic valve of any example herein, particularly example 210 or 211, wherein the valvular structure further comprises two oppositely-directed tabs extending from each movable region, wherein adjacent tabs of each two of the movable regions are joined together to form a commissure, and wherein the commissures are coupled to the main frame.

Example 214. The prosthetic valve of any example herein, particularly example 213, wherein the main frame comprises a plurality of commissure windows, and wherein the commissures are coupled to the corresponding commissure windows.

Example 215. The prosthetic valve of any example herein, particularly example 210 or 211, wherein the valvular structure further comprises at least one aperture disposed between each two adjacent movable regions.

Example 216. The prosthetic valve of any example herein, particularly example 215, wherein the at least one aperture comprises at least two apertures disposed between each two adjacent movable regions.

Example 217. The prosthetic valve of any example herein, particularly example 215 or 216, wherein the main frame comprises a plurality of commissure support struts, each commissure support strut comprising a free end, and wherein the commissure support struts extend through the corresponding apertures of the valvular structure.

Example 218. The prosthetic valve of any example herein, particularly any one of examples 187 to 209, wherein the valvular structure comprises a plurality of leaflets.

Example 219. The prosthetic valve of any example herein, particularly example 218, wherein the plurality of leaflets comprises three leaflets.

Example 220. The prosthetic valve of any example herein, particularly example 218 or 219, wherein each leaflet comprises two oppositely-directed tabs, wherein tabs of adjacent leaflets are joined together to form a commissure, and wherein the commissures are coupled to the main frame.

Example 221. The prosthetic valve of any example herein, particularly example 220, wherein the main frame comprises a plurality of commissure windows, and wherein the commissures are coupled to the corresponding commissure windows.

Example 222. The prosthetic valve of any example herein, particularly any one of examples 218 to 221, wherein the engagement portion of each leaflet comprise a strip attached to the movable portion of the leaflet.

Example 223. The prosthetic valve of any example herein, particularly example 222, wherein the strip is sutured to a cusp edge of the movable portion of the leaflet, along a stitch line.

Example 224. The prosthetic valve of any example herein, particularly example 222 or 223, wherein the strip has a uniform strip height.

Example 225. The prosthetic valve of any example herein, particularly any one of examples 187 to 224, wherein the struts of the sub-frame sections comprise angled struts and laterally extendable struts.

Example 226. The prosthetic valve of any example herein, particularly any one of examples 187 to 225, wherein at least some of the struts of the inner frame are aligned with corresponding struts of the main frame.

Example 227. The prosthetic valve of any example herein, particularly any one of examples 187 to 226, further comprising a skirt attached to a distal end of the engagement portion.

Example 228. The prosthetic valve of any example herein, particularly example 227, wherein the skirt is folded over an inflow end of the prosthetic valve, and is at least partially disposed over an outer surface of the main frame.

Example 229. The prosthetic valve of any example herein, particularly example 228, wherein the skirt is coupled to the main frame.

It is appreciated that certain features of the invention, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a main frame comprising a plurality of struts, wherein the main frame is movable between a radially compressed state and a radially expanded state;
an inner frame which is disposed radially inwardly from, and is coupled to, the main frame, the inner frame comprising a plurality of struts and a plurality of spikes extending from at least some of the plurality of struts; and
a valvular structure disposed between the main frame and the inner frame, the valvular structure comprising a movable portion and an engagement portion, wherein the movable portion is configured to regulate flow through the prosthetic valve;
wherein the plurality of spikes are engaged with the engagement portion of the valvular structure.

2. The prosthetic valve of claim 1, wherein each spike of the plurality of spikes comprises a sharp distal tip configured to penetrate into the engagement portion of the valvular structure.

3. The prosthetic valve of claim 1, wherein the engagement portion is stiffer than the movable portion.

4. The prosthetic valve of claim 1, wherein the valvular structure is formed of a unitary piece of material designed to cover the entire circumference of the prosthetic valve, between the main frame and the inner frame.

5. The prosthetic valve of claim 1, wherein the valvular structure comprises a plurality of leaflets.

6. The prosthetic valve of claim 5, wherein the engagement portion of each leaflet comprises a strip attached to the movable portion of the leaflet.

7. The prosthetic valve of claim 1, wherein the struts of the inner frame comprise angled struts and laterally extendable struts.

8. The prosthetic valve of claim 1, further comprising a skirt attached to a distal end of the engagement portion.

9. The prosthetic valve of claim 8, wherein the skirt is folded over an inflow end of the prosthetic valve, and is at least partially disposed over an outer surface of the main frame.

10. A prosthetic valve comprising:
a main frame comprising a plurality of struts defining a plurality of rungs of struts, wherein the main frame is movable between a radially compressed state and a radially expanded state;
an inner frame which is disposed radially inwardly from, and is coupled to, the main frame, the inner frame comprising a plurality of struts defining a plurality of rungs of struts, and a plurality of spikes extending from at least some of the plurality of struts; and
a valvular structure disposed between the main frame and the inner frame;
wherein the plurality of spikes are engaged with the valvular structure.

11. The prosthetic valve of claim 10, wherein the plurality of rungs of struts of the inner frame comprise: a first rung, a second rung, a third rung, and a fourth rung.

12. The prosthetic valve of claim 11, wherein the fourth rung of the inner frame is a discontinuous rung of struts.

13. The prosthetic valve of claim 12, wherein the second rung of the inner frame is a continuous rung of struts.

14. The prosthetic valve of claim 10, wherein the valvular structure comprises an engagement portion disposed between the inner frame and the main frame, and a movable portion.

15. The prosthetic valve of claim 14, wherein the engagement portion is stiffer than the movable portion.

16. The prosthetic valve of claim 14, wherein the valvular structure is formed of a unitary piece of material designed to cover the entire circumference of the prosthetic valve, between the main frame and the inner frame.

17. The prosthetic valve of claim 16, wherein the movable portion comprises a plurality of movable regions.

18. The prosthetic valve of claim 17, wherein the valvular structure further comprises at least one aperture disposed between each two adjacent movable regions.

19. The prosthetic valve of claim 18, wherein the main frame comprises a plurality of commissure support struts, each commissure support strut comprising a free end, and wherein the commissure support struts extend through the corresponding apertures of the valvular structure.

20. A prosthetic valve comprising:
a main frame comprising a plurality of struts, wherein the main frame is movable between a radially compressed state and a radially expanded state;
an inner frame assembly comprising a plurality of sub-frame sections, each sub-frame section disposed radially inwardly from, and is coupled to, the main frame, wherein each sub-frame section comprises a plurality of struts and a plurality of spikes extending from at least some of the plurality of struts; and
a valvular structure disposed between the main frame and the sub-frame sections, the valvular structure comprising a movable portion and an engagement portion, wherein the movable portion is configured to regulate flow through the prosthetic valve;
wherein the plurality of spikes are engaged with the engagement portion of the valvular structure.

21. The prosthetic valve of claim 20, wherein the sub-frame sections are laterally spaced from each other.

22. The prosthetic valve of claim 20, wherein the combined number of struts of corresponding rungs of the plurality of sub-frame sections is less than the number of struts of a corresponding rung of the main frame, for all of the rungs of the sub-frame sections.

23. The prosthetic valve of claim 20, wherein the struts of the sub-frame sections comprise angled struts and laterally extendable struts.
